(19) United States
(12) Patent Application Publication
(10) Pub. No.: US 2022/0192591 A1
(43) Pub. Date: Jun. 23, 2022

(54) BOLUS ADVISOR WITH CORRECTION BOLUSES BASED ON RISK, CARB-FREE BOLUS RECOMMENDER, AND MEAL ACKNOWLEDGEMENT

(71) Applicant: Dexcom, Inc., San Diego, CA (US)

(72) Inventor: Stephen PATEK, Charlotteville, VA (US)

(21) Appl. No.: 17/559,194

(22) Filed: Dec. 22, 2021

Related U.S. Application Data
(60) Provisional application No. 63/129,919, filed on Dec. 23, 2020.

(51) Int. Cl. A61B 5/00; A61B 5/145

(52) U.S. Cl. CPC A61B 5/486; A61B 5/4839; A61B 5/7275; A61B 5/14532

(57) ABSTRACT

Basal insulin recommendations and bolus recommendations are provided by analyzing profiles of blood glucose risk to determine whether basal or bolus amounts should be increased or decreased in consideration of the ratio of basal insulin vs. bolus insulin as a portion of total daily insulin. In some embodiments, systems and methods seek to correct systematic imbalances between rapid acting bolus and daily basal utilizing physiological cloning, which models patient diabetes data resulting from patient physiology and behavior (lifestyle and diet). In some embodiments, the systems and methods use constraints on percentage of total daily insulin attributed to basal and/or bolus. In some embodiments, optimization is performed without using patient-provided carbohydrate information.

### CROSS-REFERENCE TO RELATED APPLICATIONS

This application claims the benefit of priority to U.S. Provisional Patent Application No. 63/129,919, filed on Dec. 23, 2020, entitled “BOLUS ADVISOR WITH CORRECTION BOLUSES BASED ON RISK, CARB-FREE BOLUS RECOMMENDER, AND MEAL ACKNOWLEDGEMENT,” the contents of which are hereby incorporated by reference in their entirety.

### BACKGROUND

With the growing adoption of continuous glucose monitoring (CGM) and connected devices, the availability and reliability of glucose time-series data has increased in recent years. However, despite the availability of reliable glucose data, accurate tracking of insulin and meal data and optimized and effective timing of meal time insulin bolusing continues to be problematic for many people with diabetes resulting in poor glucose control.

Traditional clinical advice suggests a fixed percentage of basal/bolus insulin at 50/50, that is, basal insulin should be half of total daily insulin. Some more recent recommendations suggest: 40/60, 30/70, or other ratios. However, a fixed percentage is not optimal for all patients. Prior efforts to provide therapy optimization that provide basal and/or bolus recommendations to a patient have suffered from inconsistent, incomplete, or inaccurate recommendations often because the carbohydrate and/or exercise information collected/provided itself introduced inaccuracies into the data analysis.

It is with respect to these and other considerations that the various aspects and embodiments of the present disclosure are presented.

### SUMMARY

The systems and methods described herein provide basal insulin recommendations (e.g., prescriptions) and bolus recommendations (e.g., prescriptions) by analyzing profiles of blood glucose risk to determine whether basal amounts or bolus amounts should be increased or decreased in consideration of the ratio of basal insulin vs. bolus insulin as a portion of total daily insulin. Bolus insulin refers to rapid-acting insulin such as would be used for meeting insulin requirements at mealtimes or for correcting transitory high blood glucose. In some embodiments, systems and methods seek to correct systematic imbalances between rapid acting bolus and daily basal utilizing physiological cloning, which models patient diabetes data resulting from patient physiology and behavior (lifestyle and diet). In some embodiments, the systems and methods use constraints on percentage of total daily insulin attributed to basal and/or bolus. In some embodiments, optimization is performed without using patient-provided carbohydrate information.

Analysis of patients' daily insulin patterns and glycemic risk ensure an optimum balance of basal, bolus, and/or total daily insulin while minimizing hypoglycemic risk and hyperglycemic risk. An insulin recommendation system is provided that adjusts percentage basal and/or percentage bolus while reducing risks within a target range of basal/bolus ratios.

In an implementation, a method comprises: assessing a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk; quantifying daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period; determining a recommendation for one or more aspects of daily insulin of a patient based on a target range and the glycemic risk and the daily insulin relationships quantification, wherein the recommendation comprises a change in at least one of basal insulin, bolus insulin, and total daily insulin; and outputting the recommendation to a diabetes management system.

In an implementation, a system comprises: at least one processor; and a non-transitory computer readable medium comprising instructions that, when executed by the at least one processor, cause the system to: assess a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk; quantify daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period; determine a recommendation for one or more aspects of daily insulin of a patient based on a target range and the glycemic risk and the daily insulin relationships quantification, wherein the recommendation comprises a change in at least one of basal insulin, bolus insulin, and total daily insulin; and output the recommendation to a diabetes management system.

In an implementation, a system comprises: a glycemic risk assessor configured to assess a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk; an insulin relationship quantifier configured to quantify daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period; and an insulin recommender configured to determine a recommendation for one or more aspects of daily insulin of a patient based on a target range and the glycemic risk and the daily insulin relationships quantification, wherein the recommendation comprises a change in at least one of basal insulin, bolus insulin, and total daily insulin, and output the recommendation to a diabetes management system.

In an implementation, a method comprises: identifying outlier data in a daily insulin relationship pattern data set; analyzing outlier data in a daily insulin relationship pattern data set; removing the outlier data from the daily insulin relationship pattern data set; and communicating the removal of the outlier data to a diabetes management system.

In an implementation, a system comprises: at least one processor; and a non-transitory computer readable medium comprising instructions that, when executed by the at least one processor, cause the system to: identify outlier data in a daily insulin relationship pattern data set; analyze outlier data in a daily insulin relationship pattern data set; remove the outlier data from the daily insulin relationship pattern data set; and communicate the removal of the outlier data to a diabetes management system.

In an implementation, a system comprises: an insulin relationship quantifier configured to: identify outlier data in a daily insulin relationship pattern data set; analyze outlier data in a daily insulin relationship pattern data set; and remove the outlier data from the daily insulin relationship pattern data set; and an insulin recommender configured to communicate the removal of the outlier data to a diabetes management system.

In an implementation, a method comprises: identifying a variability in a daily insulin relationship pattern data set; analyzing the variability comprising analyzing data that fall at least one of above, within, or below a target range; and making adjustments to the data set based on the analyzing.

In an implementation, a system comprises: at least one processor; and a non-transitory computer readable medium comprising instructions that, when executed by the at least one processor, cause the system to: identify a variability in a daily insulin relationship pattern data set; analyze the variability comprising analyzing data that fall at least one of above, within, or below a target range; and make adjustments to the data set based on the analyzing.

In an implementation, a system comprises: at least one of an insulin relationship quantifier or an insulin recommender configured to: identify a variability in a daily insulin relationship pattern data set; analyze the variability comprising analyzing data that fall at least one of above, within, or below a target range; and make adjustments to the data set based on the analyzing.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is a high level functional block diagram of an embodiment of the invention;

FIG. 2 is a system diagram of an implementation of a bolus advisor;

FIG. 3 is a flow diagram for a method of determining a recommendation for daily insulin;

FIG. 4 is an example of a multi-graph that represents CGM data, basal insulin data, and bolus insulin data received for a patient over a time period;

FIG. 5 is an example of a CGM spaghetti graph seen in clinical reporting software for CGM systems;

FIG. 6 is a graph that shows a risk profile derived from the data of FIGS. 4 and 5, illustrating a quantified assessment of glycemic risk, both hyperglycemic risk and hypoglycemic risk;

FIG. 7 is a graph that shows one example of quantifying a daily insulin relationship pattern, including a representation of bolus insulin (y-axis) vs. basal insulin (x-axis), wherein insulin relationship patterns are shown for the data set of FIGS. 4-6 in one embodiment;

FIG. 8 is a graph that shows another example of quantifying a daily insulin relationship pattern, including a representation of the data set of FIGS. 4-6, showing bolus insulin vs. total daily insulin (TDI), wherein insulin relationship patterns are shown for the data set of FIGS. 4-6 in another embodiment;

FIG. 9 is a graph that shows another example of quantifying a daily insulin relationship pattern, including a representation of the data set of FIGS. 4-6, basal insulin vs. showing TDI, wherein insulin relationship patterns are shown for the data set of FIGS. 4-6 in yet another embodiment;

FIG. 10 is a graph that shows a representation of basal as a percentage of TDI for each day, wherein each day represents a day with sufficient data (excluding outliers) to determine a recommendation;

FIG. 11 is a flow diagram for a method of addressing outlier data in a daily insulin relationship pattern data set;

FIG. 12 is a graph of an example of outlier data within a patient in the daily insulin relationship data set indicative of outlier data in the underlying data set, which when analyzed, could represent: inaccurate data (e.g., missing basal data or bolus data) and/or misrepresentative data (inconsistent basal or bolus data);

FIG. 13 is a flow diagram for a method of addressing variability in a daily insulin relationship pattern;

FIG. 14 is a graph of an example of variability in daily insulin relationship patterns, wherein the variability may be based on days of the week, or the like;

FIG. 15 is a multi-graph of an example of CGM data that shows typical variability for a patient with type 1 diabetes;

FIG. 16 is a graph that shows both hyperglycemic and hypoglycemic risk, for example, because the profile does exceed a +/−1 threshold that may be used to identify risk, and/or because both could be quantified and related to a level of risk that may need to be addressed with basal and/or bolus adjustments;

FIG. 17 is a graph of an example daily insulin relationship quantified;

FIG. 18 is a graph of an example that shows a wide CI, showing greater variability in the data set;

FIG. 19 is a multi-graph of an example of CGM data variability for a patient with diabetes with a consistent pattern of basal injections and typical variability in bolusing;

FIG. 20 is a graph of a glycemic risk profile that shows an example of one-sided hypoglycemic risk, particularly in the morning, and which can be quantified as described in more detail herein;

FIG. 21 is a graph that shows daily insulin relationships plotted as percent bolus of TDI, wherein the daily insulin data falls well outside of the target range;

FIG. 22 is a graph that shows the daily insulin pattern confidence interval for percent basal, which is well above the target range;

FIG. 23 is a multi-graph of an example of data that indicates very consistent adherence to a basal insulin prescription;

FIG. 24 is a graph of a glycemic risk profile that identifies hyperglycemic risk exceeding a threshold of 1;

FIG. 25 is a graph of an example of a daily insulin relationship data plotted on bolus vs. TDI graph which illustrates how the data points fall outside of the target range of 45%-55% bolus of TDI;

FIG. 26 is a graph of an example that provides a recommendation to decrease percentage basal by increasing the amount of bolus because of hyperglycemia;

FIG. 27 is a multi-graph of an example where the patient has high glucose variability, indicated by numerous glucose spikes near or at 400 mg/dL;

FIG. 28 is a graph of a glycemic risk profile that identifies hyperglycemic risk above a predetermined threshold of 1;

FIG. 29 is a graph of an example daily insulin relationship quantified, with this graph plotting bolus as a percentage of TDI, the percentage of bolus is high, well outside the target range of 45-55%;

FIG. 30 is a graph that illustrates that although one data point (representing a daily insulin pattern on one day) falls within the target range, the confidence interval of the mean of the percent basal daily insulin falls clearly outside (i.e., below) of the target range for percentage basal of TDI; and

FIG. 31 shows an exemplary computing environment in which example embodiments and aspects may be implemented.

### DETAILED DESCRIPTION

The claimed subject matter is described with reference to the drawings, wherein like reference numerals are used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

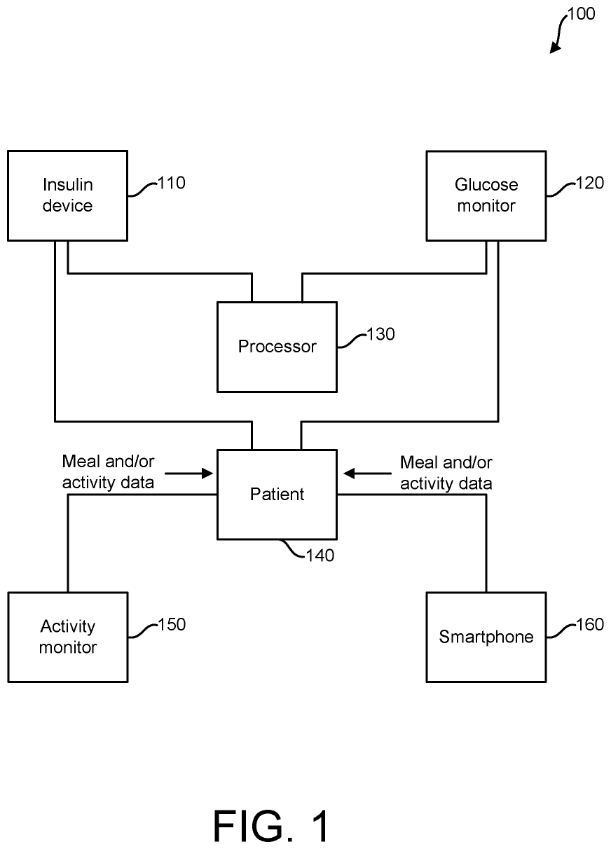

FIG. 1 is a high level functional block diagram 100 of an embodiment of the invention. A processor 130 communicates with an insulin device 110 and a glucose monitor 120. The insulin device 110 and the glucose monitor 120 communicate with a patient 140 to deliver insulin to the patient 140 and monitor glucose levels of the patient 140, respectively. The processor 130 is configured to perform the calculations and other operations and functions described further herein. The insulin device 110 and the glucose monitor 120 may be implemented as separate devices or as a single device, within a single device, or across multiple devices. The processor 130 can be implemented locally in the insulin device 110, the glucose monitor 120, or as a standalone device (or in any combination of two or more of the insulin device 110, the glucose monitor 120, or a standalone device). The processor 130 or a portion of the system can be located remotely such as within a server or a cloud-based system.

Examples of insulin devices, such as the insulin device 110, include insulin syringes, external pumps, and patch pumps that deliver insulin to a patient, typically into the subcutaneous tissue. Insulin devices 110 also includes devices that deliver insulin by different means, such as insulin inhalers, insulin jet injectors, intravenous infusion pumps, and implantable insulin pumps. An additional type of insulin device 110 is a smart insulin pen. In some embodiments, a patient will use two or more insulin delivery devices in combination, for example injecting long-acting insulin with a syringe and using inhaled insulin before meals. In other embodiments, these devices can deliver other drugs that help control glucose levels such as glucagon, pramlintide, or glucose-like peptide-1 (GLP 1).

Examples of a glucose monitor, such as the glucose monitor 120, include continuous glucose monitors that record glucose values at regular intervals, e.g., 1, 5, or 10 minutes, etc. These continuous glucose monitors can use, for example, electrochemical or optical sensors that are inserted transcutaneously, wholly implanted, or measure tissue noninvasively. Examples of a glucose monitor, such as the glucose monitor 120, also include devices that draw blood or other fluids periodically to measure glucose, such as intravenous blood glucose monitors, microperfusion sampling, or periodic finger sticks. In some embodiments, the glucose readings are provided in near real-time. In other embodiments, the glucose reading determined by the glucose monitor can be stored on the glucose monitor itself for subsequent retrieval. It is contemplated that various embodiments may be implemented with or in flash glucose monitors (FGMs).

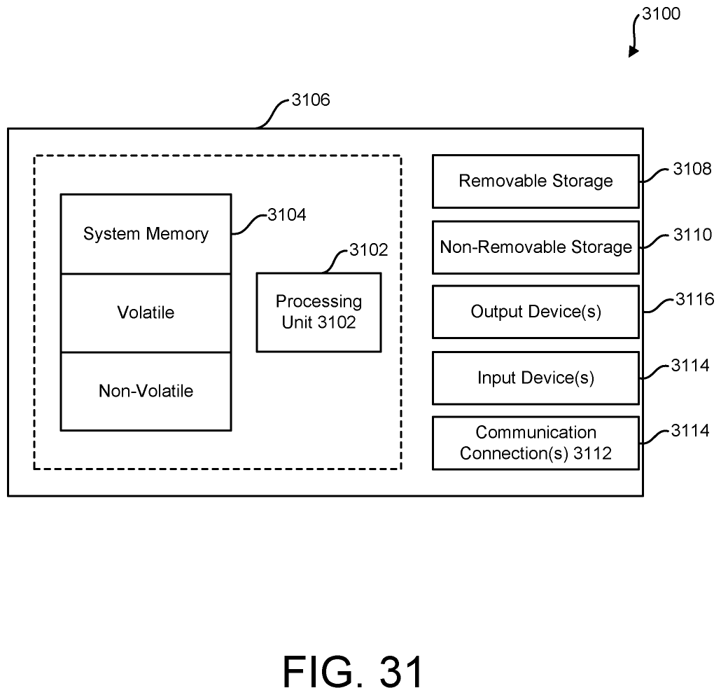

The insulin device 110, the glucose monitor 120, and the processor 130 may be implemented using a variety of computing devices such as smartphones, desktop computers, laptop computers, and tablets. Other types of computing devices may be supported. A suitable computing device is illustrated in FIG. 31 as the computing device 3100 and cloud-based applications.

The insulin device 110, the glucose monitor 120, and the processor 130 may be in communication through a network. The network may be a variety of network types including the public switched telephone network (PSTN), a cellular telephone network, and a packet switched network (e.g., the Internet). Although only one insulin device 110, one glucose monitor 120, and one processor 130 are shown in FIG. 1, there is no limit to the number of insulin devices, glucose monitors, and processors that may be supported. An activity monitor 150 and/or a smartphone 160 may also be used to collect meal and/or activity data from or pertaining to the patient 140, and provide the meal and/or activity data to the processor 130.

The processor 130 may execute an operating system and one or more applications. The operating system may control which applications are executed by the insulin device 110 and/or the glucose monitor 120, as well as control how the applications interact with one or more sensors, services, or other resources of the insulin device 110 and/or the glucose monitor 120.

The processor 130 receives data from the insulin device 110 and the glucose monitor 120, as well as from the patient 140 in some implementations, and may be configured and/or used to perform one or more of the calculations, operations, and/or functions described further herein.

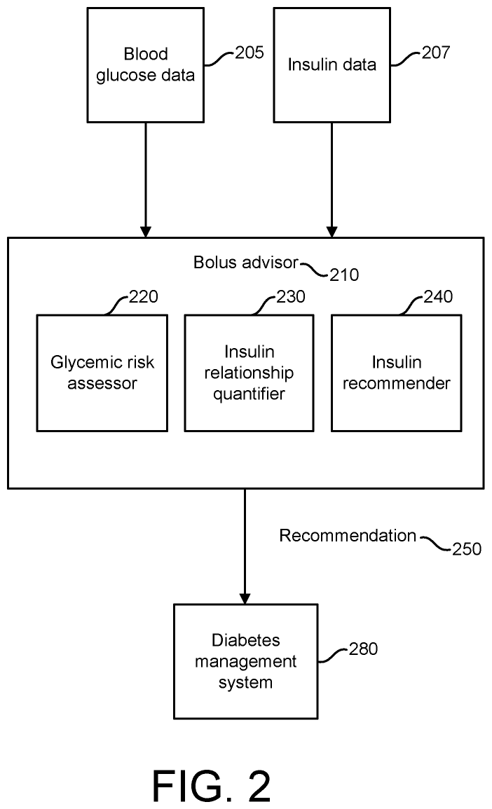

FIG. 2 is a system diagram of an implementation of a bolus advisor 210. Inputs to the bolus advisor 210 include blood glucose data 205 and insulin data 207. The blood glucose data 205 may be any diabetes data associated with a host, such as a human, and may include CGM only data, BG (blood glucose) data or other glucose or diabetes-related data, depending on the implementation. The insulin data 207 may be any insulin data associated with the host or the human, depending on the implementation.

The bolus advisor 210 comprises a glycemic risk assessor 220, an insulin relationship quantifier 230, and insulin recommender 240. The bolus advisor generates a recommendation 250 and provides the recommendation 250 to a diabetes management system 280.

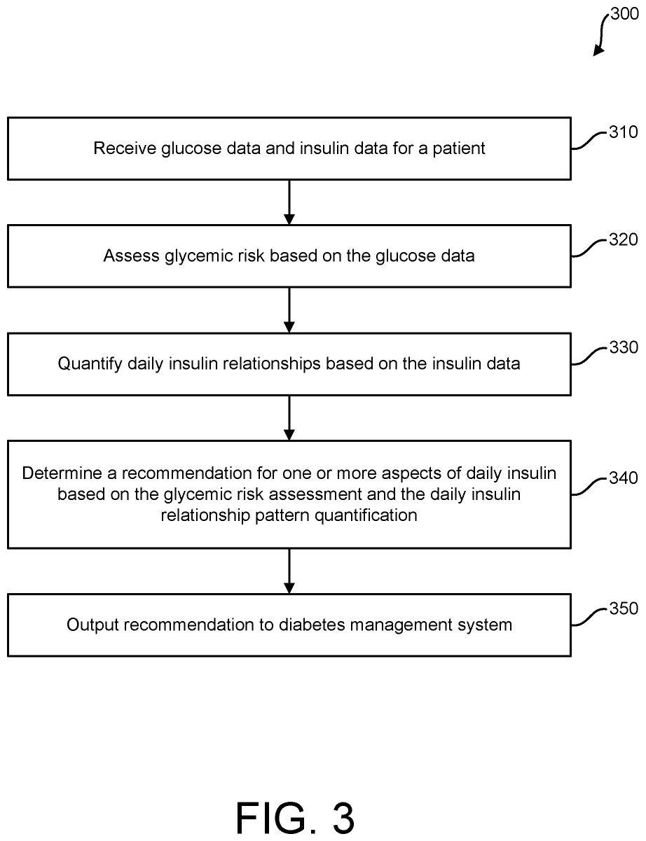

FIG. 3 is a flow diagram for a method 300 of determining a recommendation for daily insulin.

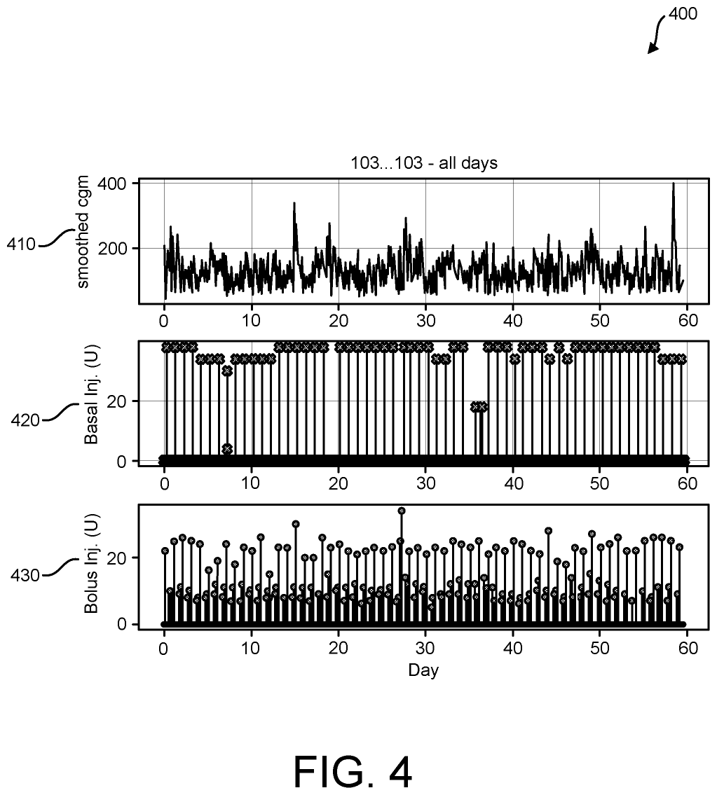

At 310, glucose data and insulin data are received for a patient. Systems and methods intake glucose data and insulin data for a time period to analyze daily insulin relationships and glycemic risk over a time period of typical patient behavior. FIG. 4 is an example of a multi-graph 400 that represents CGM data, basal insulin data, and bolus insulin data received for a patient over a time period. The glucose data and/or the insulin data may be received from the glucose monitor 120, the patient 140, the activity monitor 150, and/or the smartphone 160, in some implementations. These data typically comprise measurements of glucose levels including, for example: CGM readings, confidence readings assigned to the CGM values, self-monitoring blood glucose readings (blood glucose meter), retrospectively calibrated or corrected CGM readings, and the like. The glucose data generally encompasses a selected time period of at least one week.

The CGM graph 410 shows glucose time series data in mg/dL received for a time period of about 60 days, wherein the data was collected by a continuous glucose monitoring (CGM) system and represents blood glucose levels in the patient over the time period.

The basal inj. graph 420 shows basal insulin time series data in units of insulin received for the same time period, wherein the data was collected by a smart insulin pen and represents basal insulin injections in the patient over time.

The bolus inj. graph 430 shows bolus insulin time series data in units of insulin received for the same time period, wherein the data was collected by a smart insulin pen and represents bolus insulin injections in the patient over time.

It is contemplated that additional types of data that can be collected, including glucose and insulin data from other devices/systems as well as additional data types such as exercise and carbohydrate data, and other sensed input data and user input data. The user input data may comprise data based on meals and/or exercise and/or other activity. Meals and exercise and other activity may be explicitly ignored or not allowed in some embodiments. Additional inputs may include external process data, such as proposed basal rates and/or proposed bolus rates from an external process, which may include a pre-programmed basal profile (e.g., from an insulin pump), another AP (artificial pancreas) algorithm (e.g., AID (automated insulin delivery) system), patient-initiated insulin delivery (basal or bolus), or the like.

Some implementations use or require continuous or semi-continuous data from a continuous glucose sensor, but not necessarily in real-time. However, any glucose measurement that can be used to calculate glycemic risk as a function of time of day may be used. Retrospective or real-time data, smoothed or not smoothed data, can be used depending on the implementation. Fingerstick data may be used in some implementations, if comprehensive and preferably with carb data.

CGM data from free-living conditions is preferably used instead of collected fasting blood glucose (FBG) data. CGM data from free-living conditions over time reflects behaviors and actions in a patient's lifestyle. Conventional techniques use fasting blood glucose data, but this is not a good measure for type 1 whose daily activities vary, FBG does not assist in making decisions for much of the active insulin management decisions, and does not provide much insight into decision-making about rapid acting insulin, or in balancing basal and bolus insulin.

Implementations are used for basal/bolus/TDI optimization in intensively managed patients (those that take bolus and basal insulin), as opposed to basal titration of type 2.

CGM/insulin data received/selected must meet predetermined criteria.

Bare minimum data to assess may include CGM records and insulin records for at least (more than) a day, at least a week, or multiple weeks. Multiple weeks provide more statistically significant data, which captures a wider variety of behaviors.

Based on at how much data is available, further steps of this method may be adapted to achieve a positive result of the entire 24-hour day.

Implementations use data over a sufficient period of time that captures glucose data associated with a patient's typical lifestyle behavior, i.e., captures glucose data associated with different behavior and physiological experiences—representative of a typical lifestyle of the patient, e.g., weekends different from weekdays, typical exercise days, sleep patterns, eating habits, etc. There should be no changes to the treatment profiles in the data set.

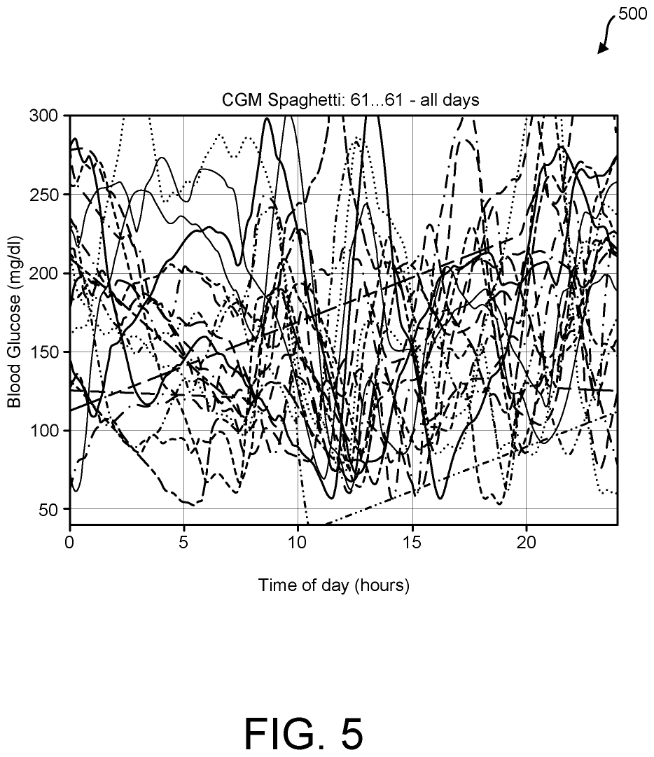

FIG. 5 is a CGM spaghetti graph 500 commonly seen in clinical reporting software for CGM systems. This exemplary graph overlays a plurality of 24-hour days of glucose data, about 60 days of glucose from the CGM graph of FIG. 4, which visualizes the variability of glucose over each 24-hour period for the consecutive days.

Because the data analysis of the systems and methods described herein are interested in daily insulin information, the start of available data may not be the start of a 24-hour time period defining a day, thus the definition of the start of the first day, and the start of a data set useful for analysis may be analyzed and determined.

It is contemplated that carbohydrate/meal information is not required for some implementations, although such information may benefit some implementations, especially where variability and/or outlier data is being analyzed.

Outlier analysis (described further herein) may be performed as part of 310.

At 320, glycemic risk may be assessed based on the glucose data. Glycemic risk may include a hyperglycemic risk and/or a hypoglycemic risk, which may be a quantification of the risk of current and future hyperglycemia and/or hypoglycemia, respectively. The glycemic risk may be calculated from the blood glucose data, which may be based on predicted glucose in some embodiments. In some embodiments, glycemic risk (e.g., hypoglycemia calculation(s) and/or hyperglycemia calculation(s)) uses prediction and/or state estimation. A glycemic risk assessor could be blood glucose (BG) risk space quantification as in low blood glucose index (LBGI)/high blood glucose index (HBGI) and/or those examples and implementations described in Patek-Actionable (U.S. Pat. No. 10,638,981, entitled “METHOD, SYSTEM AND COMPUTER READABLE MEDIUM FOR ASSESSING ACTIONABLE GLYCEMIC RISK”, inventor Stephen D. Patek, which is incorporated by reference herein in its entirety).

Risk may be assessed in terms of sample means, sample variance, time-in-range, episodes of high/low BG, low blood glucose risk, high blood glucose risk, overall risk, etc.

Any method of determining a level or risk profile of hypoglycemia and/or hyperglycemia for a patient may be utilized here, as is appreciated by one skilled in the art. Some known methods include: US20200178905A1 to Patek that describes one method to assess hypoglycemic risk, hyperglycemic risk, or both as a profile over time; US20180366223A1 to Kovatchev that describes a method of computing measures of risk for hypoglycemia and hyperglycemia, wherein low blood glucose index (LBGI) is a non-negative quantity that increases when the number and/or extent of low BG readings increases and the high blood glucose index (HBGI) is a non-negative quantity that increases when the number and/or extent of high BG readings increases; US20160239622A1 to Dunn et al. describes determining glycemic risks based on an analysis of glucose data includes visualization of hypoglycemia, variability, and hyperglycemia, all of which are incorporated by reference herein in their entireties. One example of quantifying risk profiles may be found in US20200178905A1, which is incorporated by reference herein in its entirety.

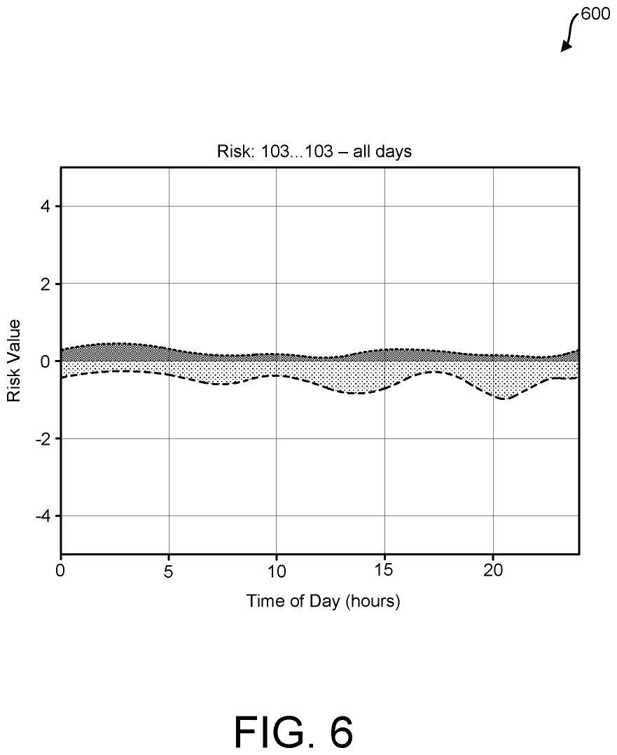

FIG. 6 is a graph 600 that shows a risk profile derived from the data of FIGS. 4 and 5, illustrating a quantified assessment of glycemic risk, both hyperglycemic risk (above the zero line) and hypoglycemic risk (below the zero line). In this example, the patient does not have a high level of either hyperglycemic risk or hypoglycemic risk as the values are well below a threshold of +/−1 over the 24-hour day.

Glycemic risk may be assessed quantitatively to determine individual risk of hyperglycemia and hypoglycemia and/or relative risk of hyperglycemia vs. hypoglycemia, e.g., if an overall weighting towards hyperglycemia vs. hypoglycemia (or vice versa) exists. In cases where risk is one-sided, meaning much higher for hypoglycemia vs. hyperglycemia (or vice versa), the relative importance of that risk is assessed and noted. In cases where risk is low overall, and generally even between hyperglycemia and hypoglycemia, a recommendation may still occur based on the quantified daily insulin relationship pattern, discussed further herein.

In some embodiments, the risk assessment is looking for a qualitative assessment of risk (yes or no), for hyperglycemic and/or hypoglycemic, for example, whether the risk profile falls outside a threshold and/or how much of the risk remains outside the threshold.

In some embodiments, the assessment quantitatively evaluates the amount of hyperglycemia and hypoglycemia overall and/or relative to the other.

The qualitative assessment(s) and/or quantitative assessment(s) may be used to determine a recommendation, i.e., whether basal and/or bolus should be adjusted, and by how much.

At 330, daily insulin relationships may be quantified based on the insulin data. In some implementations, calculate two or more aspects of daily insulin for each day over the time period of available data. Aspects of insulin include total daily insulin, total daily basal, and total daily bolus, for example.

In some implementations, by calculating each aspect of insulin, namely, at least two of total daily basal insulin (TDBasal), total daily bolus insulin (TDBolus), and total daily insulin (TDI)) for each day over a series of days, relationship patterns can be identified.

The calculated two or more aspects of insulin may be compared over a time period to quantify daily insulin relationships. A ratio or percentage of one aspect of daily insulin relative to the other aspect may be calculated. A relationship may be calculated daily, whereby daily relationship data is formed. Each aspect may be compared day by day. This may be kept as discrete data points and/or averaged over a time period.

An average of each aspect of insulin may be calculated over a time period, whereby after which the average insulin aspects may be compared and a relationship pattern formed, e.g., compute an average for each insulin aspect and then compare the averaged insulin aspects.

Mean and standard deviation may be used to calculate the confidence interval, e.g., mean and standard deviation of “percentage bolus (or basal)” (i.e., percentage of TDI attributed to bolus (or basal)) over time period may be computed. The confidence interval may be determined for the estimate of the mean. If a patient has a consistent pattern of insulin delivery from day to day, confidence interval will be narrow (e.g., a 95% confidence interval). If a patient is inconsistent, the confidence interval will be wider. Any variabilities in pattern(s) and/or outlier data may be addressed, as described further herein.

Depending on the implementation, other kinds of statistics could be used, e.g., medians or various percentiles. Each calculated aspect of insulin may be plotted on a graph, e.g., TDI vs. TDBasal, TDI vs. TDBolus, and/or TDBasal vs. TDBolus can be graphed as discrete daily data points and/or as an average or mean with a confidence interval over the time period of collected/analyzed data.

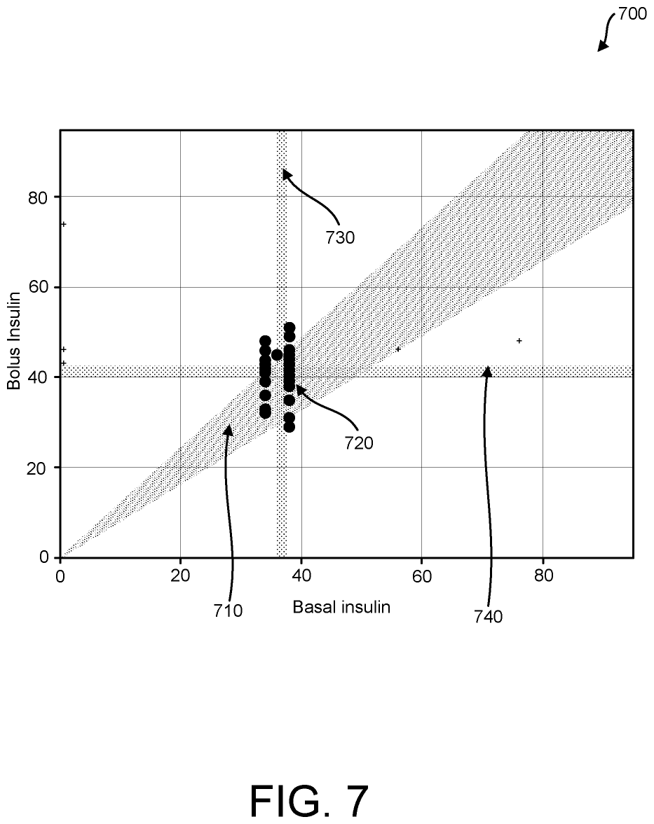

FIG. 7 is a graph 700 that shows one example of quantifying a daily insulin relationship pattern, including a representation of bolus insulin (y-axis) vs. basal insulin (x-axis), wherein insulin relationship patterns are shown for the data set of FIGS. 4-6 in one embodiment. The shaded cone 710 represents the 45-55% target range for the daily insulin relationship pattern.

The cluster of data 720 shows a trend of daily insulin that generally falls within or around the 45-55% target range. Each solid dot of the cluster of data 720 represents a daily insulin relationship data point for individual days of the patient represented in FIGS. 4-6. Each dot is a data point, wherein the x value corresponds to how much basal insulin was injected on a single day, and the y-axis value is how much bolus was injected on a particular day.

The 95% confidence for the mean basal (of all days) and 95% confidence for the mean bolus is represented by the vertical and horizontal shaded regions 730, 740, respectively. The area where the regions 730, 740 cross falls within the shaded cone 710 represents a good ratio of basal-bolus for the patient, close to 50%.

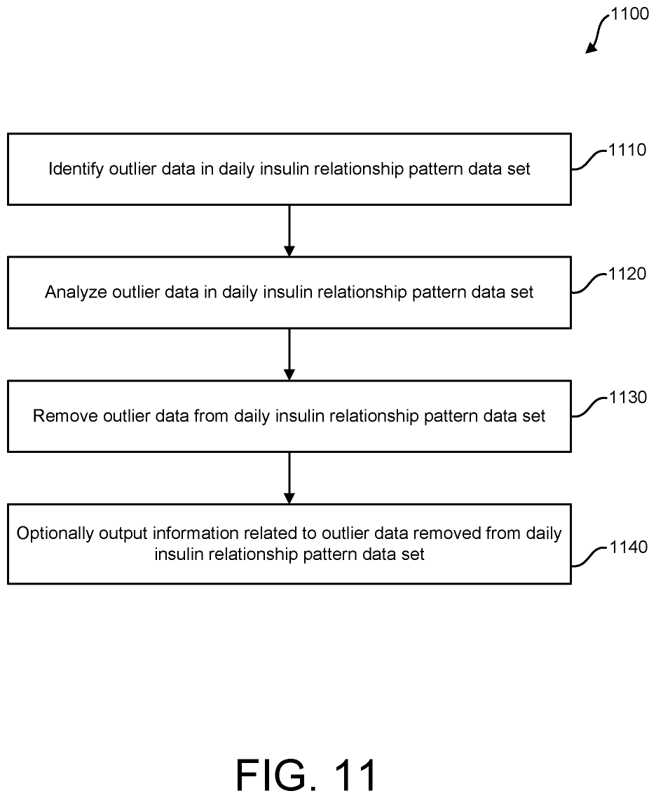

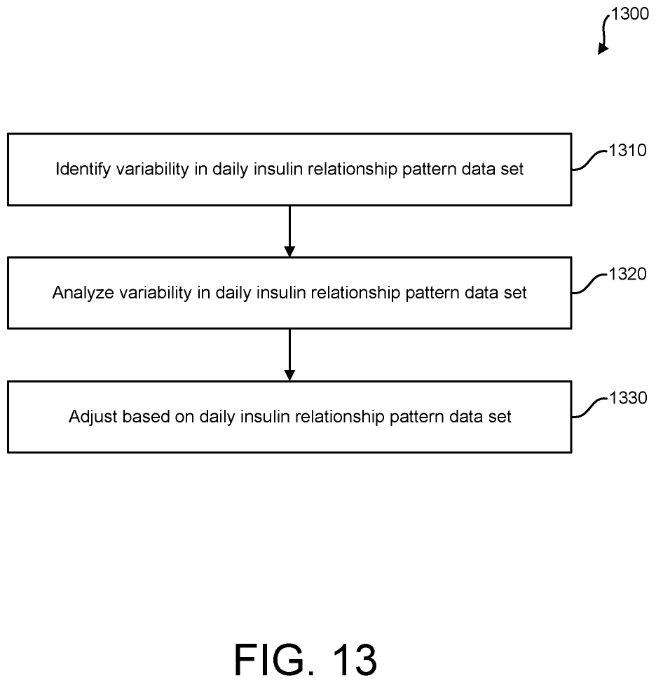

The plus marks (shown the y-axis as well as on the right side of the cone) represent outlier and/or variability data resulting from inaccurate or misrepresentative data of the patient represented in FIGS. 4-6, such as described in more detail elsewhere herein (e.g., with respect to FIGS. 11 and 13).

The plus marks on the zero basal line (i.e., the y-axis) corresponds to no basal being reported in a day. The plus marks to the right of the cone likely correspond to two basals in a day.

Outliers may be rejected as being unknown whether due to variability of behavior or inaccurate data for other reasons.

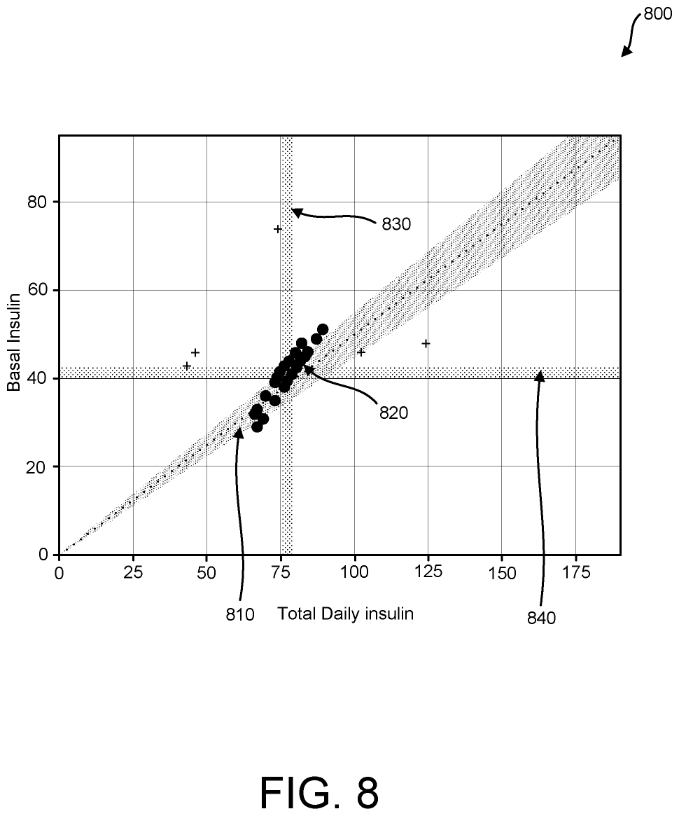

FIG. 8 is a graph 800 that shows another example of quantifying a daily insulin relationship pattern, including a representation of the data set of FIGS. 4-6, showing bolus insulin (y-axis) vs. total daily insulin (TDI) (x-axis), wherein insulin relationship patterns are shown for the data set of FIGS. 4-6 in another embodiment.

The solid dots represent daily insulin relationship data for individual days of the patient represented in FIGS. 4-6.

The plus marks (shown to the left and right side of the cone 810) represent outlier and/or variability data resulting from inaccurate or misrepresentative data of the patient represented in FIGS. 4-6, such as described in more detail elsewhere herein. y=0.5x is the centerline of the target range (percentage basal=50%).

The 95% confidence for the mean bolus and 95% confidence for the mean TDI are represented by the vertical and horizontal shaded regions 830, 840, respectively. The area where the regions 830, 840 cross falls within the shaded cone 810 represents a good ratio of bolus to TDI for the patient, close to 50/50, within target range.

The cluster of data 820 shows a trend of daily insulin that generally falls within or around the 45-55% target range.

In some embodiments, outlier data may be addressed as described in more detail elsewhere herein (e.g., with respect to FIGS. 11 and 13).

In some embodiments, data outside of a particular confidence interval (e.g., 95%) may be ignored/removed for purposes of quantifying the pattern and/or determining a change in insulin recommendations.

In this example, the outlier and/or variability analyses were triggered because there were fewer than two rapid-acting bolus and/or because the total amount of basal insulin deviated from the patient's prescription by more than 20%. Here, the three cross marks above the cone 810 correspond to days where the recorded total daily insulin equals total daily insulin, meaning no basal insulin on those days. Whether this is due to an error in record-keeping or the patient chose to take no basal insulin on those days, the data from those days is removed or ignored to address a misalignment in basal and bolus insulin overall. The two crosses with total daily insulin greater than 100 U correspond to days where the patient took substantially more than the prescribed basal insulin, again not representative of the patient's normal experience and removed or ignored by outlier and/or variability analyses.

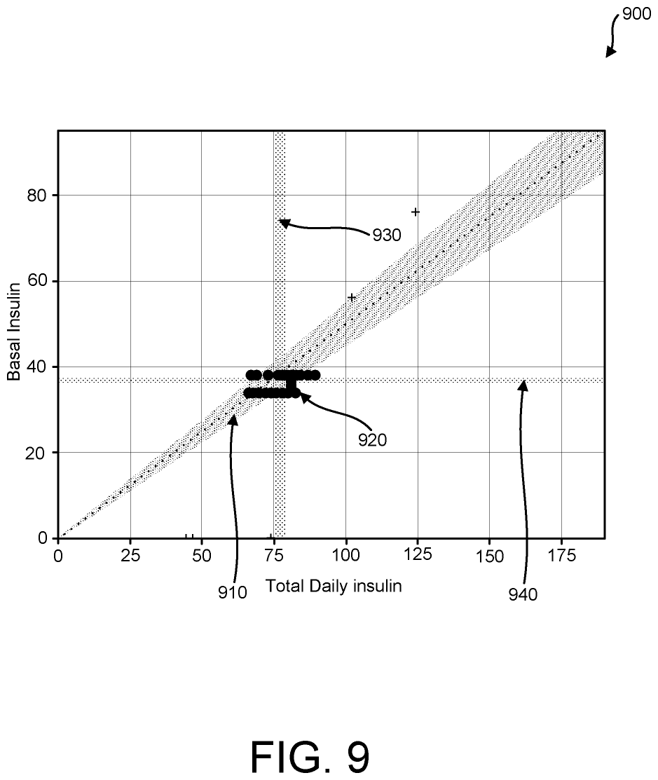

FIG. 9 is a graph 900 that shows another example of quantifying a daily insulin relationship pattern, including a representation of the data set of FIGS. 4-6, basal insulin (y-axis) vs. showing total daily insulin (TDI) (x-axis), wherein insulin relationship patterns are shown for the data set of FIGS. 4-6 in yet another embodiment.

The solid dots represent daily insulin relationship data for individual days of the patient's represented in FIGS. 4-6.

The plus marks (shown to the above the cone 910 and on the x-axis) represent outlier and/or variability data resulting from inaccurate or misrepresentative data of the patient represented in FIGS. 4-6, such as described in more detail elsewhere herein.

The cluster of data 920 shows a trend of daily insulin that generally falls within or around the 45-55% target range. The 95% confidence for the mean basal and 95% confidence for the mean TDI are represented by the vertical and horizontal shaded regions 930, 940, respectively. The area where the regions 930, 940 cross falls within the shaded cone 910 represents a good ratio of basal to TDI for the patient, close to 50%, within target range.

Variability and/or outliers in the data may be addressed as described in more detail elsewhere herein (e.g., with respect to FIGS. 11 and 13).

In this example, the data from a given day was considered to be an outlier and/or not representative if there were fewer than two rapid-acting bolus or if the total amount of basal insulin deviated from the patient's prescription by more than 20%. Here, the three cross marks on the basal=0 line (i.e., the x-axis) correspond to days where the recorded total daily insulin equals total daily insulin, meaning no basal insulin on those days. Whether this is due to an error in record-keeping or the patient chose to take no basal insulin on those days, the data from those days should not be used to assess a misalignment in basal and bolus insulin overall. The two crosses with total daily insulin greater than 100 U correspond to days where the patient took substantially more than the prescribed basal insulin, again not representative of the patient's normal experience.

A group of data above or below the cone 910 may represent a superset of varying patterns. In this case, data above the cone 910 may represent, for example cases wherein a patient has a varied lifestyle (e.g., sporadic exercising).

In some embodiments, multiple daily insulin patterns may be identified, such as week vs. weekend patterns as described in more detail elsewhere herein.

Data output from this step 330 may include the daily insulin relationships, one or more patterns of daily insulin relationships, and average of daily insulin relationships, one or more patterns of daily insulin relationships, outlier data, and/or variability data.

At 340, a recommendation is determined for one or more aspects of daily insulin based on the glycemic risk assessment and the daily insulin relationship pattern quantification that indicates whether basal or bolus should be increased, decreased, both, or neither.

A recommendation may be determined when a daily insulin pattern is outside a target range, and a resulting recommendation (how to adjust basal/basal/TDI) may be determined based on glycemic risk.

In some implementations, a recommendation may be considered when a daily insulin pattern is outside a target range and is confirmed (whether to adjust basal/basal/TDI) based on glycemic risk, wherein the recommendation is determined based on the amount the daily insulin pattern is outside the range and the quantitative or qualitative assessment of risk.

In some implementations, a recommendation may be determined when a daily insulin pattern is within a target range, but a glycemic risk does not meet one or more criteria. For example, where a hyperglycemic risk is high, increasing both basal and bolus may be considered to reduce risk while maintaining daily insulin aspects within a target range.

The quantification of a daily insulin relationship pattern discussed above informs whether an adjustment in daily insulin aspects should be made. Namely, when the daily insulin relationship pattern falls outside the target range, an adjustment should be considered. For example, when a percentage basal is above the target range, it should be lowered whereas when percentage basal is below target range, it should be increased.

Data output from 320 and/or 330 may be analyzed and compared to a target range, which may be static or dynamic, and which may be adaptive to a patient (e.g., based on patient settings or patient patterns) and/or dependent on the day (e.g., weekend vs. weekday).

The target range defines a range of acceptable values for the daily insulin pattern data from 330, which assists in the determination of whether the daily insulin pattern is within an acceptable range and/or whether an increase or decrease in basal insulin, bolus insulin, and/or total daily insulin may be recommended.

The target range could be lifestyle-dependent, genetic, and/or based on stage of diabetes, depending on the implementation. The target range could be arbitrary (e.g., selected by a clinician or patient or set as a default), or could be based on variability assessment or other analysis of patient data.

In contrast to prior art that utilizes a fixed basal/bolus ratio for insulin optimization, systems and methods described herein do not attempt to target a “one size fits all” approach, such as fixed 50/50 or 40/60 ratio for a patient. Rather, the determining step allows for a range of ratios that achieve good glycemic outcomes. In other words, the systems and methods described herein evaluate the glucose data to decide whether the patient's daily insulin relationships are within a range of allowable basal/bolus ratios, which are appropriate for the patient's actual needs for improved glycemic control. Furthermore, methods and systems described herein allow for adaptation of target insulin ratios over time.

Daily insulin relationship data that falls within the target range may not be subject to any further analysis and the processing may proceed to an output step, wherein the patient may be informed that no change is recommended at this time. However, processing may also continue to the adjustment determination based on the glycemic profile of the patient. In other words, when a patient falls within the target range, but the patient's data indicates a risk of hypoglycemia or hyperglycemia above a predetermined threshold, the systems and methods described herein may continue to determine and recommend an adjustment to a daily insulin aspect that allows for reduced glycemic risk and maintains the daily insulin pattern within the target range.

In some situations, although basal/bolus is outside the target range, no recommendation may be determined, for example, when the assessment of glycemic risk shows little or no glycemic risk (determined quantitatively and/or qualitatively as described further herein). The systems and methods described herein may alternatively recommend small changes in basal and/or bolus when a target range is prioritized, either by the software or a clinician utilizing the software, depending on the implementation.

The assessment of glycemic risk discussed above informs how the adjustment should be made (to basal, to bolus, or to both). Namely, which daily aspect(s) should be increased or decreased.

Systems and methods described herein are guided by glycemic risk to determine whether basal should be adjusted or boluses should be adjusted, or both, with or without a change to TDI.

Daily insulin relationship data outside the target range are subject to an adjustment determination based on the patient's glycemic risk profile. That is, the determination of a recommendation to increase and/or decrease any one or more daily insulin aspect is based on an analysis of hyperglycemic risk and/or hypoglycemic risk determined by, or in accordance with, the patient's glucose data, depending on the implementation.

For example, in a case where glycemic risk is low overall, but percentage basal should be increased, but the glycemic risk is generally even between hyperglycemia and hypoglycemia, both an increase to basal and a decrease to bolus, together, increase the percentage basal. In this case, because the risk is not one-sided, the recommendation hits both basal and bolus but has the net effect of increasing percentage basal.

The glycemic risk assessment and/or daily insulin quantification may further determine by how much the adjustments should be made, for example, 5% increase to basal and 5% decrease to bolus. Actual insulin amounts in units may be calculated as well, as may be appreciated by one skilled in the art.

Some exemplary recommendations include but are not limited to:

When the daily insulin pattern falls above a target range, the determined adjustment may include: an increase in daily basal, a decrease in daily bolus, or both, dependent on the patient's glycemic risk profile.

When the daily insulin pattern falls below a target range, the determined adjustment may include: a decrease in daily basal, an increase in daily bolus, or both, dependent upon the patient's glycemic risk profile.

When the daily insulin pattern does not clearly fall within or outside of the range, for example, wherein the confidence interval of the pattern is half-in/half-above the upper boundary of the target range, which shows tendency to have too much bolus, but is not so significant to recommend change, some implementations may evaluate glycemic risk, message the patient, and/or provide feedback to address outliers and/or variability in the data, as described in more detail elsewhere herein (e.g., with respect to FIGS. 11 and 13).

In some cases, when the daily insulin pattern is outside a target range, but the glycemic risk profile is within a threshold, then no recommendation may be determined and/or the system may determine an adaptive target range for the patient and/or query the patient about daily insulin patterns and custom target ranges.

In some cases, when the daily insulin pattern is within the target range, but the glycemic risk profile is outside a threshold, there may be a problem with the patient's prescription, but the problem is not really one of basal/bolus being misaligned. In such cases, recommend a consultation with the patient's doctor or other medical professional to discuss sources of out-of-range blood glucose data (e.g., significant hyperglycemia/hypoglycemia) other than basal and bolus insulin ratios.

In some implementations, the systems, methods, and/or logic described herein either increase the basal or decrease the bolus, or vice versa, in a situation where the glycemic risk is clearly one-sided (depending on whether the one-sided risk is either hypoglycemia or hyperglycemia but not both). Alternatively, when the hyperglycemia and hypoglycemia are evenly matched, then the systems, methods, and/or logic adjust both (e.g., increases or decreases basal or bolus depending on which one needs to be increased or decreased).

Any adjustment (increase or decrease) to one or more aspects of daily insulin (TDBasal, TDBolus, TDI) may be determined by glycemic risk and the daily insulin relationship pattern (i.e., relative to a target range). In other words, when glucose data indicates glycemic risk above/below a predetermined threshold, adjustments may be simulated for one or more percentage increases/decreases in one or more of the aspects of daily insulin using the midpoint, outer boundaries, or any area of the range as adjustment target as may be appreciated by one skilled in the art. In addition, the adjustment selected may be a percentage increase/decrease informed by the target range, as described above, a set point, and/or any other data available from the patient (e.g., lifestyle of patient, outlier, variability of patient data, relative risk tolerance, etc.).

The following guidelines may be followed when the daily insulin pattern falls outside the target range; however, other guidelines may be useful as well depending on the implementation and/or situation.

When hyperglycemia risk is above a threshold and daily insulin pattern is above a target range, then increase percentage TDI from basal.

When hypoglycemia risk is above a threshold and daily insulin pattern is above a target range, decrease percentage TDI from bolus.

When hyperglycemia risk is above a threshold and daily insulin pattern is below a target range, decrease percentage TDI from basal.

When hypoglycemia risk is above a threshold and daily insulin pattern is below a target range, increase percentage TDI from bolus.

When there exists hypoglycemia risk without hyperglycemic risk, or vice versa, TDI may be decreased or increased, respectively.

A change in TDI may be alone or in combination with a change in basal/bolus.

In some cases, wherein an increase or a decrease is applied only to one aspect of daily insulin, TDI may increase or decrease respectively therewith. In some cases, TDI is determined to be adjusted.

In some embodiments, TDI is allowed to vary while changing basal/bolus ratios; however, a limit of allowable TDI change may be applied.

If TDI falls outside an acceptable change limit, the determined adjustment may be re-evaluated relative to further adjustments of other aspects of daily insulin until additional criteria are met, e.g., criteria for glycemic risk, criteria for daily insulin patterns, and further criteria such as allowable TDI, doctor recommended basal insulin ranges, specific criteria associated with available units of insulin (e.g., rounding to an integer), limits associated with dosing methods, and the like.

In some cases, a combination of (i) reducing basal and (ii) increasing bolus may be determined, for example, based on the risk of hyperglycemia relative to the risk of hypoglycemia.

TDI may be a variant and the systems and methods may identify, e.g., the 25-75 percentile range and flag situations where the patient is experiencing/reporting unusual amounts of total insulin in a day.

In some situations, a data set, and an associated daily insulin pattern, may not be sufficiently consistent to accurately determine an adjustment using the systems and methods described herein without additional insights about the patient's daily behavioral patterns, which can happen for a variety of reasons, e.g., because of differences in needs/activities/behavior/eating behaviors from day to day. For example, TDI or total daily basal (TDB) needs may not be the same from day to day. Sometimes, patient data is highly variable or even misrepresentative. In these cases, addressing outlier data and/or variability in the data may be performed prior to, in combination with, after, or iterative in a feedback look with the adjustment determination.

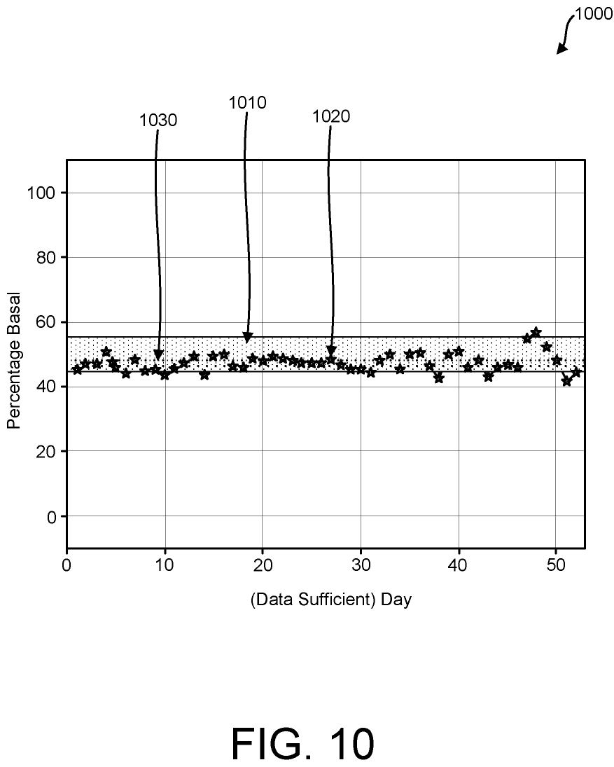

FIG. 10 is a graph 1000 that shows a representation of basal as a percentage of TDI (y-axis) for each day (x-axis), wherein each day represents a day with sufficient data (excluding outliers) to determine a recommendation. The shaded band 1010 represents the 45-55% target range for basal as a percentage of TDI. The x-axis is day numbers where there was sufficient quality data for the day to be used in the calculation (not necessarily consecutive, but all days where the percentage basal data values and the percentage bolus data values are complete and not outlying).

The stars 1020 are data points that represent basal as a percentage of TDI for a single day. The stars 1020 have a mean value that is right in the middle of the dotted rectangular band 1030 and 95% CI is the confidence that the mean is within that band.

The dotted rectangular band 1030 in middle of the target range is the 95% confidence interval for the estimate of the mean of the data points, and the band is narrow and well within the target range. In this example, the very narrow band falling with the target range indicates that the actual mean percentage basal is very close to the sample average (with 95% confidence). It is also an indication that the daily variation of percentage basal is quite small and, here, very close to 50% basal of TDI, meaning 95% of time it is very close to that range of values—for percentage basal of TDI in the day. This exemplifies a patient in good control with good 50/50 basal-bolus ratio.

This is an example of someone who may not require a recommended adjustment in the daily insulin. Further examples described in more detail elsewhere herein show other scenarios of typical patients with diabetes that would benefit from the systems and methods described herein to adjust basal and/or bolus values on a daily basis.

At 350, a recommendation (or recommendations) is output to a diabetes management system. The recommendation(s) may be in the form of a report, a command, or a signal or instructions to an insulin delivery system, a therapy optimization algorithm, or the like. Additionally or alternatively, the recommendation(s) may be provided to the patient, a doctor or other medical professional, an administrator, or any device, system, and/or algorithm that manages diabetes. In some implementations, a message may be sent to the patient, doctor, medical professional, administrator, etc. regarding any outlier data, variability in data, a determined recommendation with a query or observation, making a recommendation with or without additional context (e.g., regarding variability), prompting for feedback to re-run algorithm, or the like.

In some embodiments, a determined adjustment (e.g., a numerical adjustment or recommendation) is rounded to an integer value or other applicable unit.

When outputted to an insulin delivery system, a determined bolus adjustment may be implemented by making proportional changes to carb ratio and/or correction factor, for example.

Another implementation of a determined adjustment could be to be increase or decrease basal and/or bolus by a percentage rather than a specific amount, such as 10% increase or decrease.

A determined recommendation could be output with messaging for adjusting the insulin conditional on an environmental or behavioral consistency, for example regarding exercise behavior or eating patterns.

In some cases, do not recommend a change, but message the patient about their significant hyperglycemia risk and/or hypoglycemia risk despite achieving a reasonable balance between basal insulin and bolus insulin and recommend a consultation with their doctor to discuss other sources of out-of-range blood glucose.

It is contemplated that the systems, methods, and/or algorithms described herein may be implemented as a module of an the open-loop therapy titration algorithm or system.

FIG. 11 is a flow diagram for a method 1100 of addressing outlier data in daily insulin relationship pattern data set. In some situations, a data set, and associated daily insulin pattern, may be skewed by outlier data, for example, patient data that is missing or misrepresentative. Examples of compromised data integrity include failure to record (or record in timely fashion) basal events and/or bolus events, perhaps resulting from a technical failure in a “connected” insulin pen or pump. Examples of outlier activity include running a marathon and climbing a mountain, where the patient may intentionally deviate significantly from prescribed basal insulin for one or more days. Addressing of outliers may be performed before, during, and/or after any of the steps of the methods described herein, for example as a data scrubbing and/or confirmation step and/or as a feedback loop within one or more steps of the method.

At 1110, outlier data is identified in a daily insulin relationship pattern data set, which may be inaccurate or contain misrepresentative data points. Outlier data includes inaccurate or misrepresentative data that skews pattern analysis, which may result in an incorrect insulin adjustment recommendation. It is contemplated that some cases may exist in which there are a lot of outliers, and the non-outliers demonstrate a very wide range of daily percentage-basals such that, despite there being glycemic risk, no equalization recommendation can be made.

Inaccurate data includes missing data and/or inaccurately labeled data. Inaccurate data includes any data in the underlying data set used to identify includes missing data (no bolus insulin reported in a day), improperly time-stamped data, or otherwise corrupted or bad data, empty fields, zero values, etc. Examples include a patient forgetting to enter bolus information and/or data is not transferred properly from the insulin delivery device to the software running the daily insulin relationship evaluation algorithm.

In an example use case of an MDI (multiple daily injection) patient that uses two different insulin pens for basal insulin delivery and bolus insulin delivery, syncing and/or manual data entry of delivered basal amounts and bolus amounts may suffer from user-driven errors or technology-related errors.

Misrepresentative data includes data that may be accurate but resulting from outlier behavior in eating or activity patterns. Data may be an exception or may be driven by outlier behavior. Examples of outlier behavior include fasting and binging, running out of insulin, running a marathon, other extenuating circumstances, etc. which is data that skews daily pattern analysis.

Identifying outlier data includes identifying any inaccurate or misrepresentative data described above or as appreciated by one skilled in the art.

At 1120, the outlier data is analyzed. Analyzing outlier data in a daily insulin relationship pattern includes analyzing the data set underlying the analysis to determine whether enough good data exists to make a recommendation and/or determine an adjustment.

In this manner, a consistent pattern in basal-to-bolus ratios may be ensured. Without consistent patterns, adjustment determination may be inaccurate or irrelevant.

Generally speaking, any day where percentage basal is below a predetermined threshold or greater than another predetermined threshold is a day where there is probably either (i) missing information about insulin delivery or (ii) or there is something non-representative about the day (e.g., patient ran out of basal insulin, ran a marathon, etc.)

Analyzing outlier data may be as straightforward as ignoring or removing data from the data set where there are no basal doses or no bolus doses. The analysis of daily insulin patterns of the systems and methods described herein includes, in some implementations, analysis of at least two of the daily insulin aspects described further herein. The data associated with those at least two of the daily insulin aspects includes a complete data set for both basal and bolus (and thus total daily insulin). Without daily data for basal or bolus, the systems and methods described herein may output inaccurate recommendations.

In some embodiments, the data set is analyzed for statistical consistency to ensure a consistent pattern may be obtained.

In some embodiments, the systems and methods analyze the data set for a minimum number of days that meet a threshold of consistency. It may be ensured that the actual ratio computed for that day lie within some reasonable wide range of possible ratios (e.g., somewhere between 10% and 90%), and/or that a certain number of days meet a criterion.

Analysis may evaluate consistency and/or variability between days—e.g., statistical deviation limited by threshold, e.g., if ratio has a 95% confidence interval wider than allowable range for TDI.

In some implementations, daily TDB and TDI values are collected from the historical record, dropping non-compliant days.

In some implementations, CGM data for at least a week is used for pattern determination.

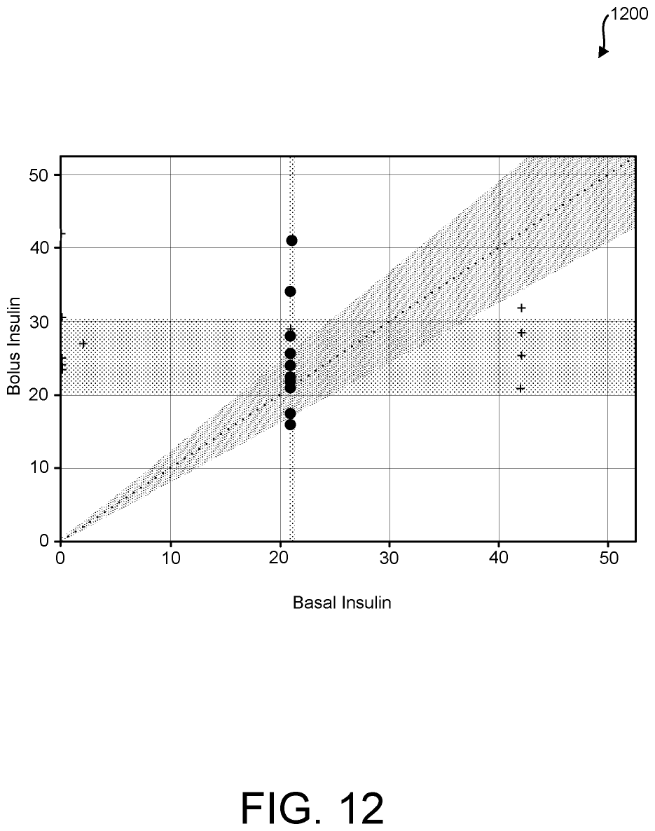

FIG. 12 is a graph 1200 of an example of outlier data within a patient in the daily insulin relationship data set indicative of outlier data in the underlying data set, which when analyzed, could represent: inaccurate data (e.g., missing basal or bolus data) and/or misrepresentative data (inconsistent basal or bolus data). In this case, there are several days where the basal insulin is twice as large as the median basal value, and there are several corresponding days where basal insulin is zero. This may be reflective of a situation where the patient is late in taking his/her basal insulin on a given day (creating a 0-basal day) and then additionally taking their regularly prescribed basal dose at the usual time (creating a 2x basal day). In some embodiments, days of this sort could be removed or ignored by the systems and methods described herein.

At 1130, the outlier data is removed from the daily insulin relationship pattern data set. In some implementations, the TDI is evaluated over a range of data to determine whether data is within predetermined range. A TDI range from data set may be used to limit TDI range for adjustment determination method steps, using identification of (and removal of) outliers from the data set.

At 1140 (optional), information is output (e.g., to a patient, a doctor, an administrator, a storage device, a report, etc.) related to the outlier data that was removed from the daily insulin relationship pattern data set. The removal of outlier data may be communicated to a diabetes management system and/or fed back into one or more of the methods described herein. In some implementations, the patient and/or doctor may be notified (e.g., by message) to determine a root cause or confirm accuracy or representation of the data.

FIG. 13 is a flow diagram for a method 1300 of addressing variability in daily insulin relationship pattern. The process of addressing variability may be triggered when the data fall outside one or more predetermined criteria, e.g., by comparing the quantified insulin relationship pattern data to the target range. Criteria include whether and/or how much of the data fall above, within, and/or below the target range.

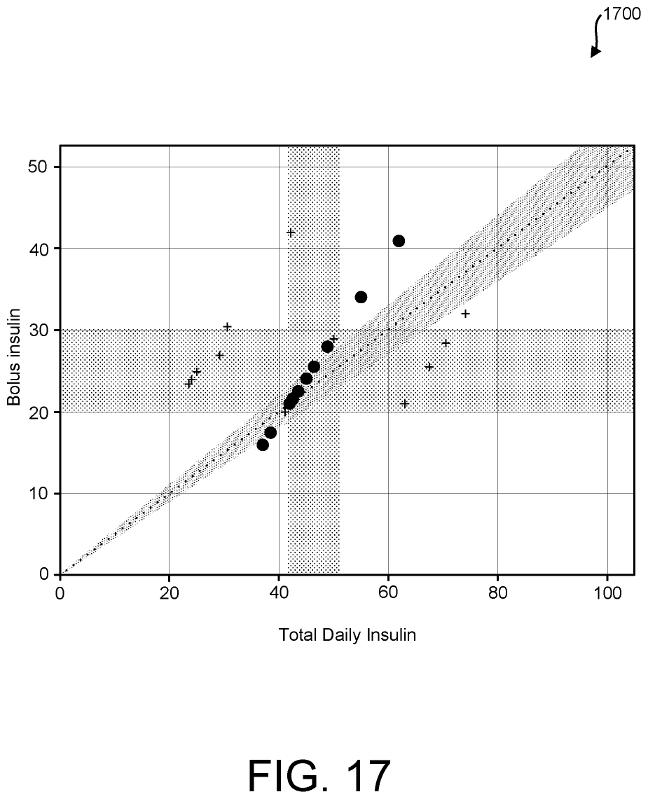

In one example, if the 95% confidence interval for the mean completely encompasses the target range, credible days above range exist and credible days below range exist. In this case, instead of just indicating “not clearly out of range”, an indicator may indicate something like “there is no clear pattern to your basal/bolus mix”. FIG. 17 is a graph 1700 of an example of this (e.g., an example daily insulin relationship quantified).

In some situations, a data set, and associated daily insulin pattern, may be a superset of two or more subsets of patterns associated with data variability, for example, patient data that is variable over time.

Addressing data variability may be performed before, during, and/or after any of the steps of the method 300 of FIG. 3 and/or the method 1100 of FIG. 11, for example as a feedback loop (e.g., sending back the subsets to the determination step within one or more steps of the method).

At 1310, identify variability in a daily insulin relationship pattern data set.

In some embodiments, more than one pattern may be represented by the data set. In general, there may not be a single consistent or systematic adjustment/determination to make. Additionally or alternatively, there is not actually an adjustment to make when the patient is already self-adjusting for these behavioral differences.

Variability in patterns may result from a wide confidence interval (outside a threshold), wherein differing analysis of time periods, days of weeks definitions, etc. may result in multiple differing patterns.

Variability may be identified by analyzing individual data points and/or evaluating a mean and/or standard deviation against one or more criteria, for example.

The process of identifying a variability may include evaluating a superset of data, the data set described elsewhere herein, to identify one or more subsets of data associated with a different daily insulin pattern.

At 1320, analyze the variability in the daily insulin relationship pattern data set. The process of analyzing the data may include analyzing the data that fall above, within, and/or below the target range.

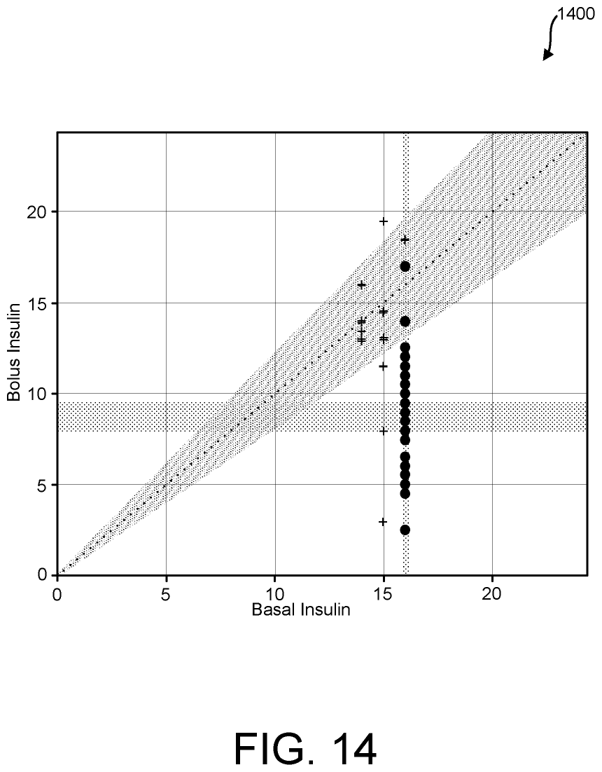

FIG. 14 is a graph 1400 of an example of variability in daily insulin relationship patterns, wherein the variability may be based on days of the week, or the like. For example, the patient may have a varied lifestyle (e.g., night shift, alternating exercise pattern, weekend eating binges, or the like) and require a variable solution.

Implementations of systems and/or methods may query a patient and/or find a way to pull out subsets of patterns and/or analyze additional data, if available, related to eating or activity.

Analyzing variability in daily insulin relationship pattern includes analyzing the data set underlying the analysis to determine whether enough good data exists to make a recommendation and/or determine an adjusted basal recommendation and/or bolus recommendation for the entire data set or not.

In some situations, a data set, and associated daily insulin pattern, may not be sufficiently consistent to accurately evaluate using the systems and methods described herein without additional insights about the patient's daily behavioral patterns, which can happen for a variety of reasons, e.g., because of differences in needs/activities/behavior/eating behaviors from day to day. For example, TDI or total daily basal (TDB) needs may not be the same from day to day. Sometimes, patient data is highly variable due to normal behavioral patterns or abnormal behavioral patterns.

At 1330, adjustments are made based on the analyzed variability in the daily insulin relationship pattern data set. Adjusting may include adjusting definitions of data (e.g., start/end of 24-hour time period), which may result in different sleeping patterns, working patterns (night shift) and/or dividing data into two or more distinct data sets/patterns (e.g., weekdays vs. weekends).

Accordingly, the systems and methods described herein allow for adaptation of TDI and/or basal/bolus ratio over time. The techniques are used, in some implementations, when the TDI over a range of data is within a reasonable range. Processes may be implemented to try to make this fit, e.g., change definition of day and/or run a moving average.

TDI with daily ratios may be computed. Outliers may be removed in a feedback loop. The definition of day may be changed within a feedback loop, and may be based on a CGM trace. With a plurality of TDIs with daily ratios, these could be provided as continuous, average, moving average. In some implementations, weekdays may be differentiated from weekends (or workdays vs. off days). In some implementations, the end of a day may be defined.

Examples are now described.

### EXAMPLE 1

This example illustrates a situation where (i) historical data for a patient indicates some exposure to hypoglycemia risk and hyperglycemia risk but (ii) the ratio of basal insulin to total daily insulin is not clearly outside of the 45%-55% range, ultimately resulting a “no change” recommendation.

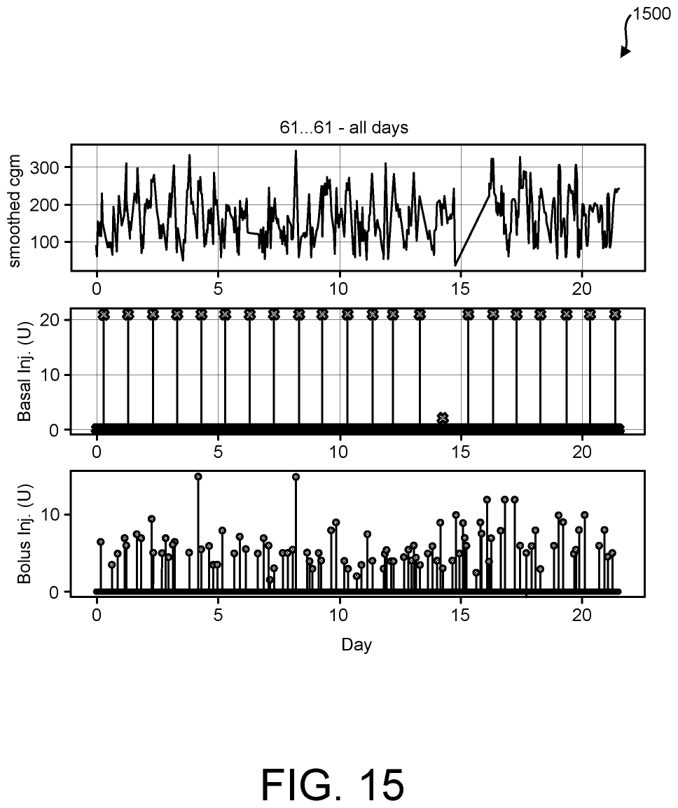

FIG. 15 is a multi-graph 1500 of an example of CGM data that shows typical variability for a patient with type 1 diabetes. The CGM data shows typical variability for a patient with type 1 diabetes. The basal injections are generally consistent, with the exception of day 15, which would trigger outlier analysis (removal) prior to the step of assessing glycemic risk based on the data. Also noteworthy is that the bolusing pattern is fairly sporadic, but not unusual for a patient with type 1 diabetes with a varied lifestyle.

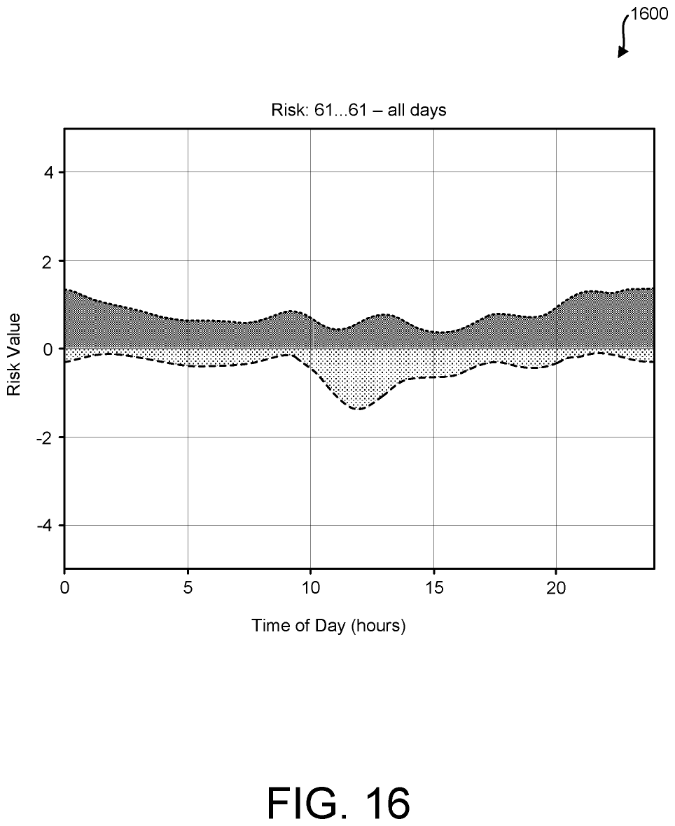

FIG. 16 is a graph 1600 that shows both hyperglycemic risk and hypoglycemic risk, for example, because the profile does exceed a +/−1 threshold that may be used to identify risk, and/or because both could be quantified and related to a level of risk that may need to be addressed with basal and/or bolus adjustments.

FIG. 17 is a graph 1700 of an example daily insulin relationship quantified. However, because so many outliers and so much variability exist in the data, it may be unclear how to make a recommendation. At least some of the outliers appear to have occurred due to basal values either not being reported, or perhaps taken to late/early and counted for a different day.

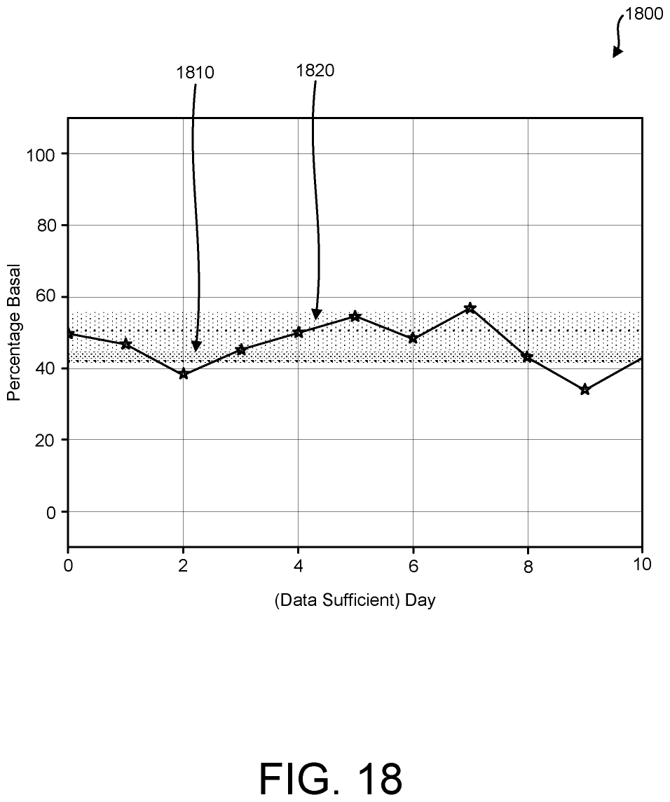

FIG. 18 is a graph 1800 of an example that shows a wide CI (as compared to FIG. 10, for example), showing greater variability in the data set. The CI zone 1810 falls at least partly within the target range zone 1820, and thus the mean estimate of the daily insulin pattern may fall within the target range. Additionally, in this exemplary data set, numerous outliers exist, as well as non-outliers that demonstrate a very wide range of daily percentage-basal such that, despite there being some glycemic risk, no insulin recommendation is determined here.

### EXAMPLE 2

This example illustrates a situation where (i) historical data for a patient indicates significant exposure to hypoglycemia risk and (ii) the ratio of basal insulin to total daily insulin is significantly above 45%-55% range (equivalently the ratio of bolus insulin to total daily insulin is significantly below the 45%-55% range). While the insulin ratio can be normalized by either decreasing basal insulin or increasing bolus insulin (or both), because of the risk of hypoglycemia, the system/method/apparatus here opts to decrease basal insulin, thereby decreasing total daily insulin.

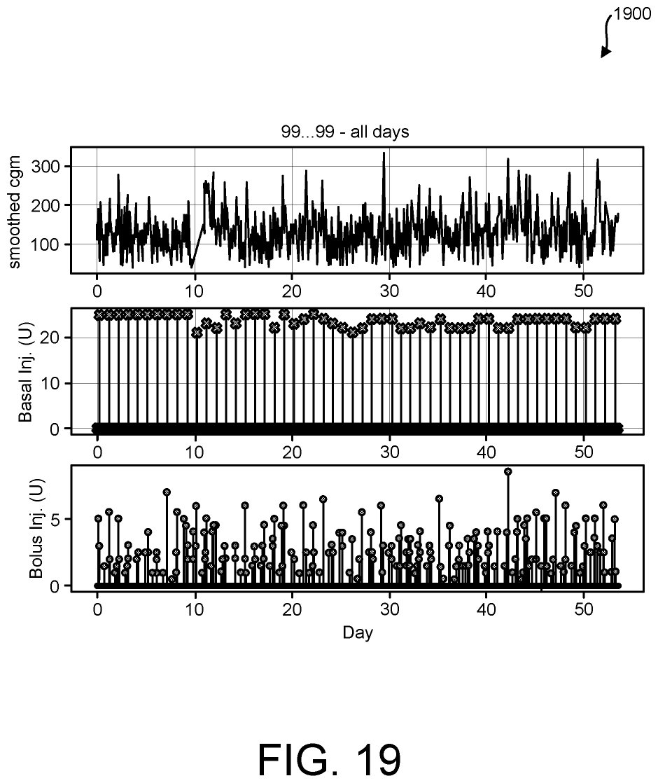

FIG. 19 is a multi-graph 1900 of an example of CGM data variability for a patient with diabetes with a consistent pattern of basal injections and typical variability in bolusing. Compared to EXAMPLE 1, FIG. 19 shows some variability in the daily basal insulin dose. Under more extreme variability in the daily basal dose, an exemplary output to a diabetes management system might recommend the patient be more consistent in basal/bolus behavior strategy and/or in reporting consistency in order to utilize the recommender.

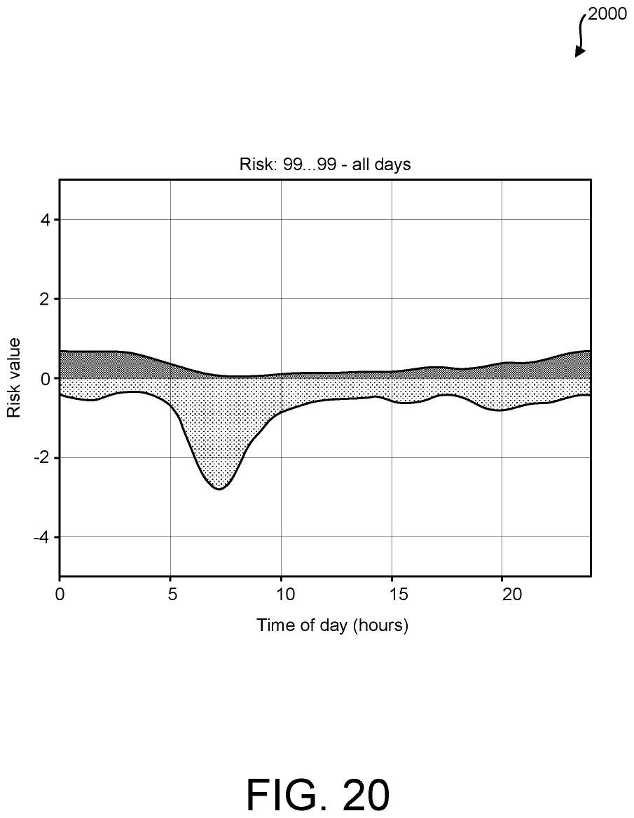

FIG. 20 is a graph 2000 of a glycemic risk profile that shows an example of one-sided hypoglycemic risk, particularly in the morning, and which can be quantified as described in more detail elsewhere herein.

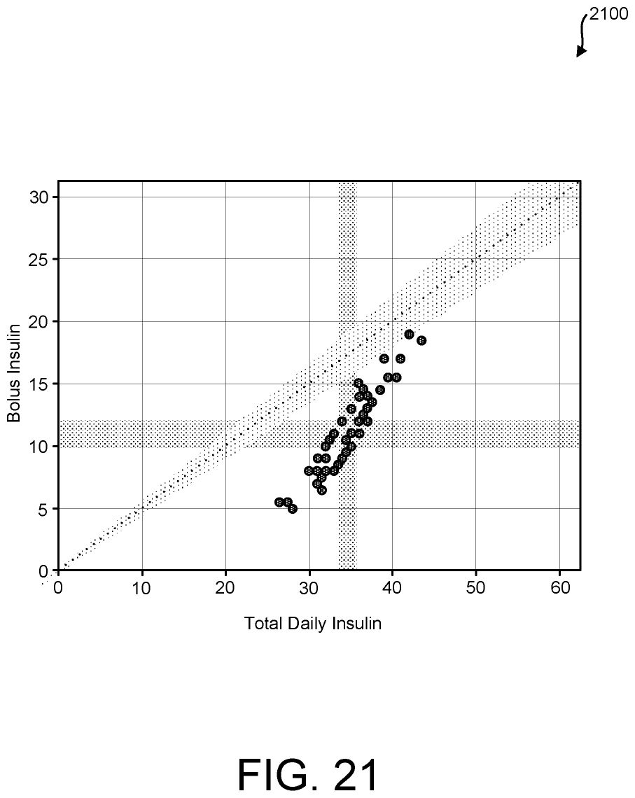

FIG. 21 is a graph 2100 that shows daily insulin relationships plotted as percent bolus of TDI, wherein the daily insulin data falls well outside of the target range. Specifically, percent bolus is consistently lower than expected (relative to the target range).

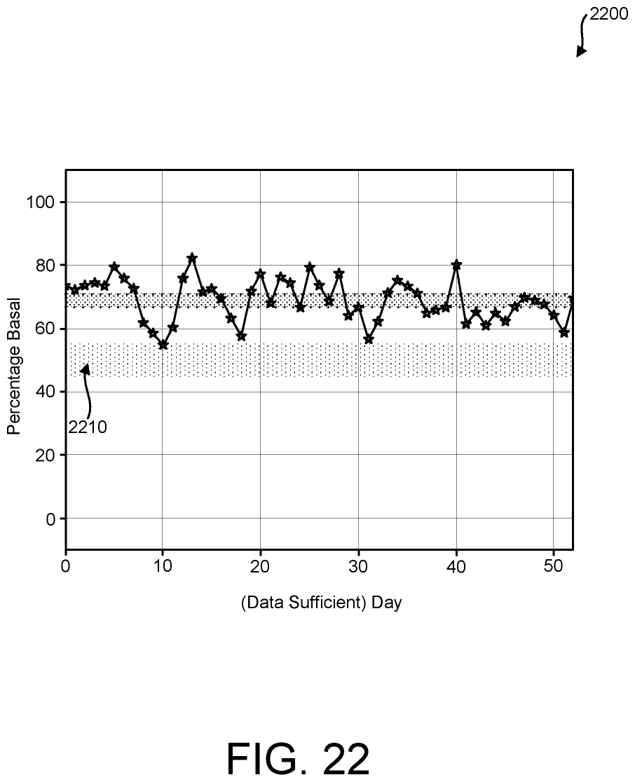

FIG. 22 is a graph 2200 that shows the daily insulin pattern confidence interval for percent basal, which is well above the target range 2210. In this exemplary data set, the systems and methods determine a recommendation to decrease percentage basal by decreasing amount of basal based on the one-sided hypoglycemia risk assessed by the risk profiling (FIG. 20) and the quantified daily insulin pattern (FIG. 21).

Exemplary output to a diabetes management system could include a specific amount, such as “decrease basal by 10%”.

### EXAMPLE 3

This example illustrates a situation where (i) historical data for a patient indicates significant exposure to hyperglycemia risk and (ii) the ratio of basal insulin to total daily insulin is significantly above 45%-55% range (equivalently the ratio of bolus insulin to total daily insulin is significantly below the 45%-55% range). While the insulin ratio can be normalized by either decreasing basal insulin or increasing bolus insulin (or both), because of the risk of hyperglycemia, the system/method/apparatus here opts to increase bolus insulin, thereby increasing total daily insulin.

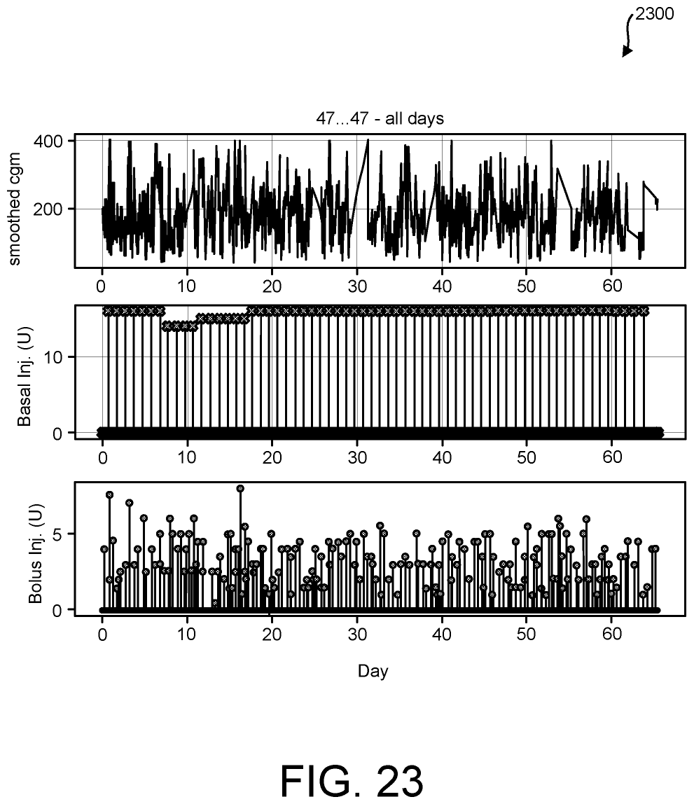

FIG. 23 is a multi-graph 2300 of an example of data that indicates very consistent adherence to a basal insulin prescription, except for the week after Day 7 after experiencing severe hypoglycemia the patient may have decided to attenuate basal insulin to avoid a repeat occurrence.

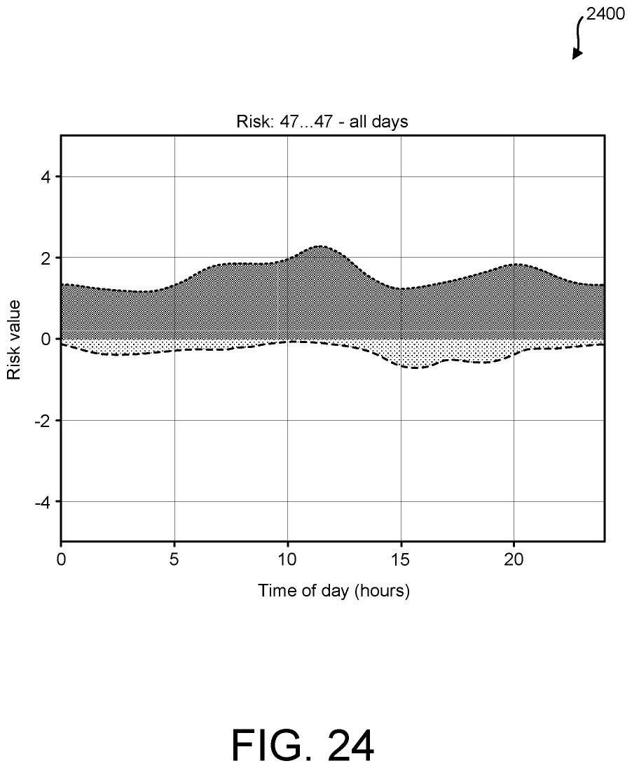

FIG. 24 is a graph 2400 of a glycemic risk profile that identifies hyperglycemic risk exceeding threshold of 1. The data indicate that the patient is consistently managing insulin to avoid hypoglycemia, sometimes referred to as a “hypo fearing” individual.

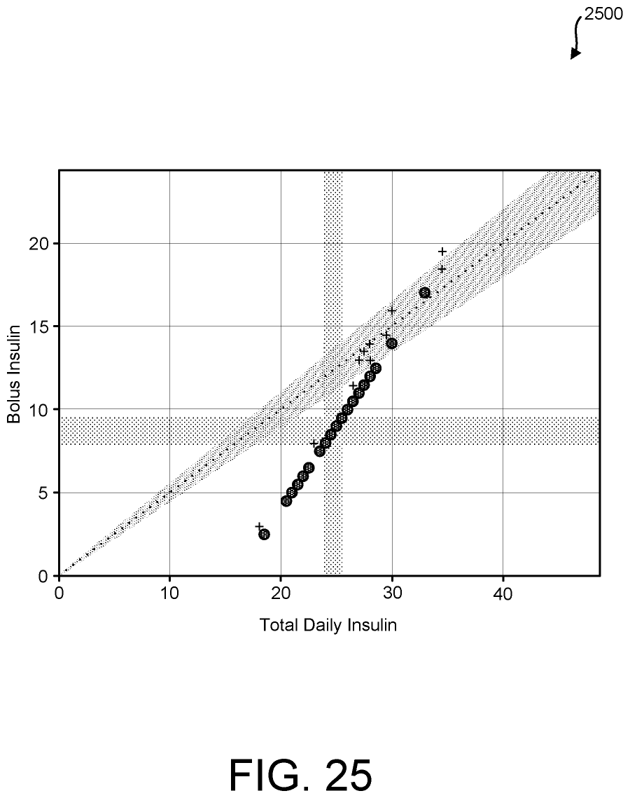

FIG. 25 is a graph 2500 of an example of a daily insulin relationship data plotted on bolus vs. TDI graph which illustrates how the data points fall outside of the target range of 45%-55% bolus of TDI.

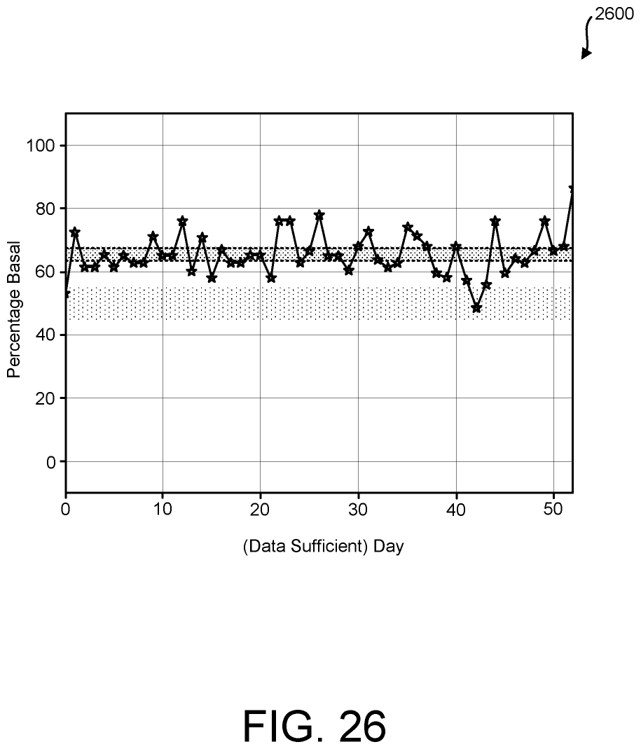

FIG. 26 is a graph 2600 of an example that provides a recommendation to decrease percentage basal by increasing the amount of bolus because of hyperglycemia. When hyperglycemia is the assessed risk (one-sided), more insulin is needed in the patient's system and because the daily insulin pattern indicated too much basal, the logic of the systems/method is designed to increase insulin and decrease percentage basal, e.g., by increasing bolus.

Exemplary output to a diabetes management system may be increase all boluses (except for the basal boluses) by 10% or to program a parameter change to the carb ratio and/or correction factor that will output an increased bolus from a bolus calculator used at meals and/or for corrections.

### EXAMPLE 4

This example illustrates a situation where (i) historical data for a patient indicates significant exposure to hyperglycemia risk and (ii) the ratio of basal insulin to total daily insulin is significantly below 45%-55% range (equivalently the ratio of bolus insulin to total daily insulin is significantly above the 45%-55% range). While the insulin ratio can be normalized by either increasing basal insulin or decreasing bolus insulin (or both), because of the risk of hyperglycemia, the system/method/apparatus here opts to increase basal insulin, thereby increasing total daily insulin.

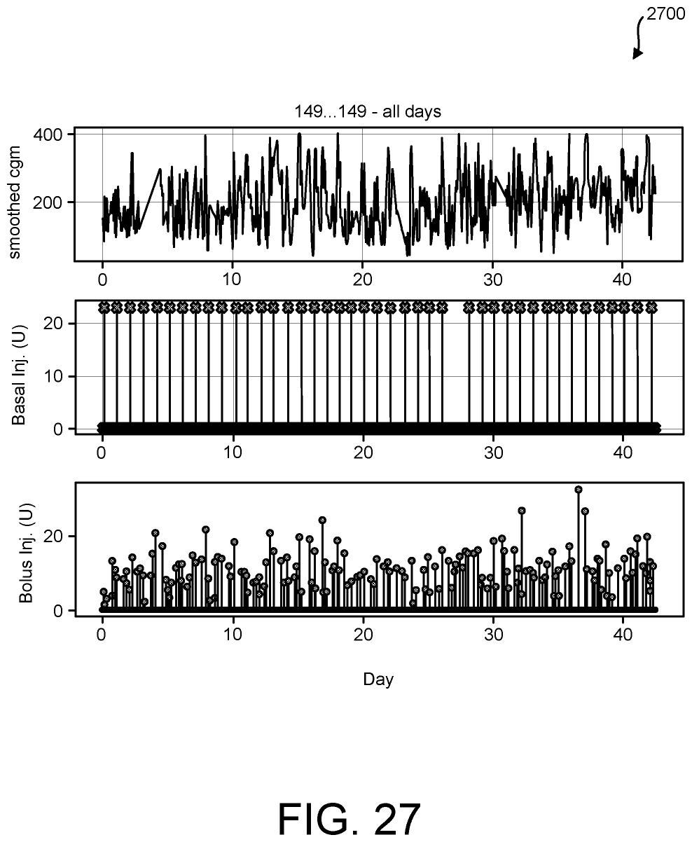

FIG. 27 is a multi-graph 2700 of an example where the patient has high glucose variability, indicated by numerous glucose spikes near or at 400 mg/dL. The patient appears to take their basal insulin of about 23 Units once a day very consistently, and the variation in insulin throughout the day can be attributed to bolus insulin delivery.

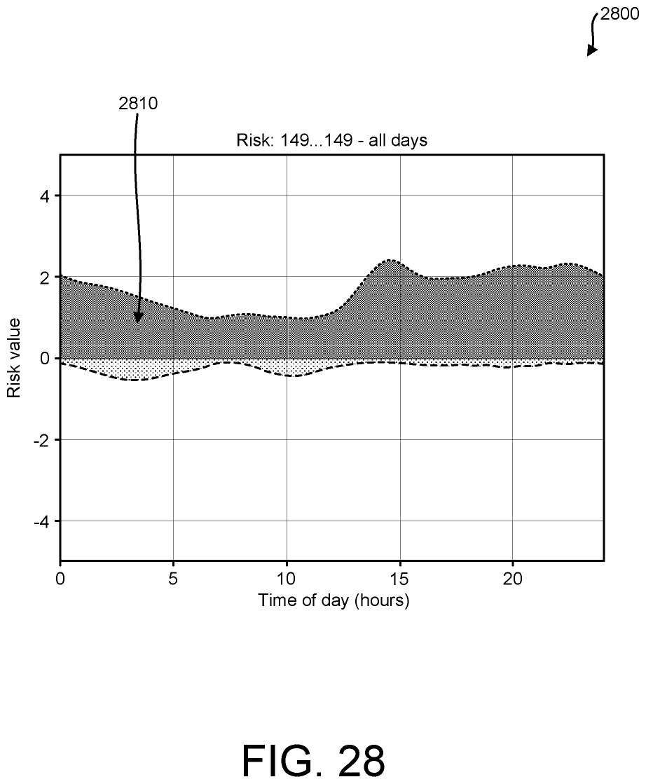

FIG. 28 is a graph 2800 of a glycemic risk profile that identifies hyperglycemic risk above a predetermined threshold of 1 (here even exceeding 2). When hyperglycemia risk is quantified, e.g., area under the curve of all values above 0 (shading 2810) correlating with glucose above a threshold, it can be seen that the risk assessment is highly correlated with hyperglycemia risk. The amount above the hyperglycemia glucose threshold is clearly greater than amount below the hypoglycemia threshold, showing a heavy weighting toward hyperglycemia risk. None of the hypoglycemia risk profile falls outside a threshold of 1. The issue this patient is experiencing is related to hyperglycemia, rather than hypoglycemia.

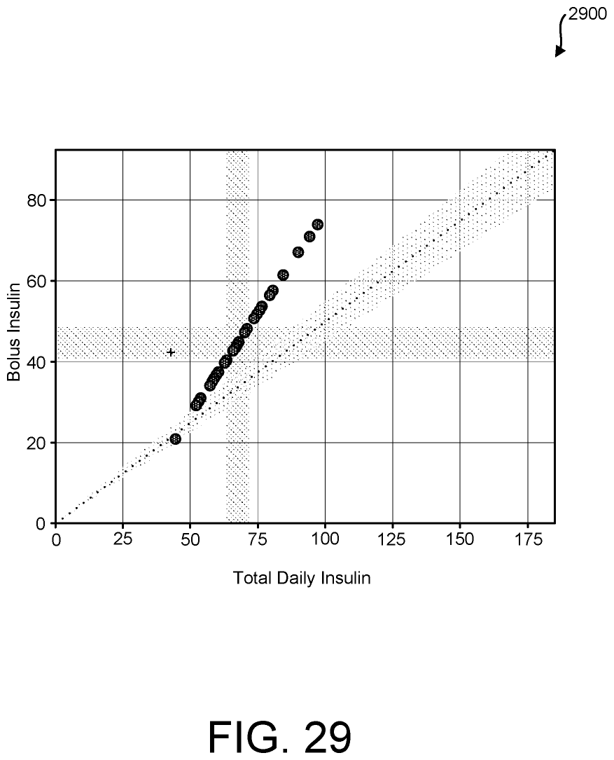

FIG. 29 is a graph 2900 of an example daily insulin relationship quantified, with this graph plotting bolus as a percentage of TDI, the percentage of bolus is high, well outside the target range of 45-55%. The corollary states then that basal as a percentage of TDI insulin is too low, outside the target range of 45-55%.

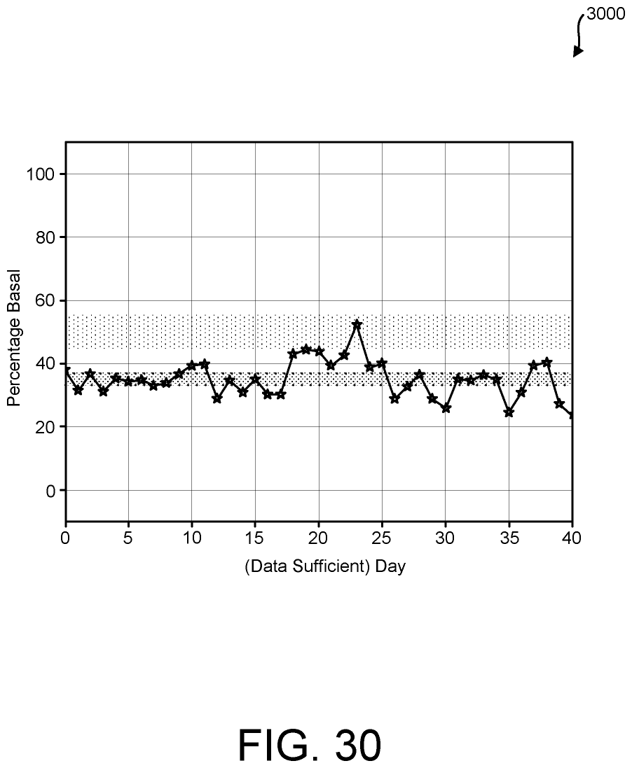

FIG. 30 is a graph 3000 that illustrates that although one data point (representing a daily insulin pattern on one day) falls within the target range, the confidence interval of the mean of the percent basal daily insulin falls clearly outside (i.e., below) of the target range represented by the shaded area 3010 for percentage basal of TDI. In this exemplary data set, the systems and methods described herein determine a recommendation to increase percentage basal by increasing amount of basal based on the hyperglycemic risk identified in FIG. 28 and the daily insulin relationship pattern identified in FIG. 29.

Exemplary output to a diabetes management system could include a numerical recommendation such as “increase daily basal by 10%” or a qualitative recommendation, such as “increase basal, please see physician for detailed guidelines”.

FIG. 31 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 31, an exemplary system for implementing aspects described herein includes a computing device, such as computing device 3100. In its most basic configuration, computing device 3100 typically includes at least one processing unit 3102 and memory 3104. Depending on the exact configuration and type of computing device, memory 3104 may be volatile (such as random access memory (RAM)), non-volatile (such as read-only memory (ROM), flash memory, etc.), or some combination of the two. This most basic configuration is illustrated in FIG. 31 by dashed line 3106.

Computing device 3100 may have additional features/functionality. For example, computing device 3100 may include additional storage (removable and/or non-removable) including, but not limited to, magnetic or optical disks or tape. Such additional storage is illustrated in FIG. 31 by removable storage 3108 and non-removable storage 3110.

Computing device 3100 typically includes a variety of computer readable media. Computer readable media can be any available media that can be accessed by the device 3100 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 3104, removable storage 3108, and non-removable storage 3110 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 3100. Any such computer storage media may be part of computing device 3100.

Computing device 3100 may contain communication connection(s) 3112 that allow the device to communicate with other devices. Computing device 3100 may also have input device(s) 3114 such as a keyboard, mouse, pen, voice input device, touch input device, etc. Output device(s) 3116 such as a display, speakers, printer, etc. may also be included. All these devices are well known in the art and need not be discussed at length here.

In an implementation, a method comprises: assessing a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk; quantifying daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period; determining a recommendation for one or more aspects of daily insulin of a patient based on a target range and the glycemic risk and the daily insulin relationships quantification, wherein the recommendation comprises a change in at least one of basal insulin, bolus insulin, and total daily insulin; and outputting the recommendation to a diabetes management system.

Implementations may include some or all of the following features. The method further comprises receiving the glucose data and the insulin data for the patient prior to assessing the glycemic risk and quantifying the daily insulin relationships. The glycemic risk is a quantification of the risk of current and future hyperglycemia and/or hypoglycemia. Assessing the glycemic risk uses at least one of prediction and state estimation. The glycemic risk is assessed in terms of at least one of sample means, sample variance, time-in-range, episodes of high/low BG, low blood glucose risk, high blood glucose risk, and overall risk. The method further comprises generating a risk profile that illustrates a quantified assessment of glycemic risk. The aspects of daily insulin comprise at least one of total daily insulin, total daily basal, or total daily bolus. The method further comprises identifying relationship patterns based on the aspects of daily insulin for each day over a series of days. Quantifying the daily insulin relationships based on insulin data further comprises calculating at least one of a ratio and a percentage of one aspect of daily insulin relative to another aspect. The method further comprises removing outlier data from at least one of the glucose data and the insulin data prior to at least one of assessing the glycemic risk and quantifying the daily insulin relationships. Determining the recommendation is performed when a daily insulin pattern is outside the target range, and wherein the recommendation is based on the glycemic risk. Determining the recommendation is performed when a daily insulin pattern is outside the target range and is confirmed based on the glycemic risk, wherein the recommendation is based on the amount the daily insulin pattern is outside the range and at least one of a quantitative assessment of risk and a qualitative assessment of risk. Determining the recommendation is performed when a daily insulin pattern is within the target range and when a glycemic risk fails to meet at least one criteria. The change comprises an increase or a decrease in an amount of at least one of basal insulin, bolus insulin, and total daily insulin. The recommendation is in the form of a report, a command or signal or instructions to an insulin delivery system, or a therapy optimization algorithm.

In an implementation, a system comprises: at least one processor; and a non-transitory computer readable medium comprising instructions that, when executed by the at least one processor, cause the system to: assess a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk; quantify daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period; determine a recommendation for one or more aspects of daily insulin of a patient based on a target range and the glycemic risk and the daily insulin relationships quantification, wherein the recommendation comprises a change in at least one of basal insulin, bolus insulin, and total daily insulin; and output the recommendation to a diabetes management system.

Implementations may include some or all of the following features. The computer readable medium further comprises instructions that, when executed by the at least one processor, cause the system to receive the glucose data and the insulin data for the patient prior to assessing the glycemic risk and quantifying the daily insulin relationships. The glycemic risk is a quantification of the risk of current and future hyperglycemia and/or hypoglycemia. Assessing the glycemic risk uses at least one of prediction and state estimation. The glycemic risk is assessed in terms of at least one of sample means, sample variance, time-in-range, episodes of high/low BG, low blood glucose risk, high blood glucose risk, and overall risk. The computer readable medium further comprises instructions that, when executed by the at least one processor, cause the system to generate a risk profile that illustrates a quantified assessment of glycemic risk. The aspects of daily insulin comprise at least one of total daily insulin, total daily basal, or total daily bolus. The computer readable medium further comprises instructions that, when executed by the at least one processor, cause the system to identify relationship patterns based on the aspects of daily insulin for each day over a series of days. Quantifying the daily insulin relationships based on insulin data further comprises calculating at least one of a ratio and a percentage of one aspect of daily insulin relative to another aspect. The computer readable medium further comprises instructions that, when executed by the at least one processor, cause the system to remove outlier data from at least one of the glucose data and the insulin data prior to at least one of assessing the glycemic risk and quantifying the daily insulin relationships. Determining the recommendation is performed when a daily insulin pattern is outside the target range, and wherein the recommendation is based on the glycemic risk. Determining the recommendation is performed when a daily insulin pattern is outside the target range and is confirmed based on the glycemic risk, wherein the recommendation is based on the amount the daily insulin pattern is outside the range and at least one of a quantitative assessment of risk and a qualitative assessment of risk. Determining the recommendation is performed when a daily insulin pattern is within the target range and when a glycemic risk fails to meet at least one criteria. The change comprises an increase or a decrease in an amount of at least one of basal insulin, bolus insulin, and total daily insulin. The recommendation is in the form of a report, a command or signal or instructions to an insulin delivery system, or a therapy optimization algorithm.

In an implementation, a system comprises: a glycemic risk assessor configured to assess a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk; an insulin relationship quantifier configured to quantify daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period; and an insulin recommender configured to determine a recommendation for one or more aspects of daily insulin of a patient based on a target range and the glycemic risk and the daily insulin relationships quantification, wherein the recommendation comprises a change in at least one of basal insulin, bolus insulin, and total daily insulin, and output the recommendation to a diabetes management system.

Implementations may include some or all of the following features. The glycemic risk assessor is further configured to receive the glucose data and the insulin data for the patient prior to assessing the glycemic risk and quantifying the daily insulin relationships. The glycemic risk is a quantification of the risk of current and future hyperglycemia and/or hypoglycemia. Assessing the glycemic risk uses at least one of prediction and state estimation. The glycemic risk is assessed in terms of at least one of sample means, sample variance, time-in-range, episodes of high/low BG, low blood glucose risk, high blood glucose risk, and overall risk. The insulin relationship quantifier or the insulin recommender is further configured to generate a risk profile that illustrates a quantified assessment of glycemic risk. The aspects of daily insulin comprise at least one of total daily insulin, total daily basal, or total daily bolus. The insulin relationship quantifier is further configured to identify relationship patterns based on the aspects of daily insulin for each day over a series of days. Quantifying the daily insulin relationships based on insulin data further comprises calculating at least one of a ratio and a percentage of one aspect of daily insulin relative to another aspect. The glycemic risk assessor is further configured to remove outlier data from at least one of the glucose data and the insulin data prior to at least one of assessing the glycemic risk and quantifying the daily insulin relationships. Determining the recommendation is performed when a daily insulin pattern is outside the target range, and wherein the recommendation is based on the glycemic risk. Determining the recommendation is performed when a daily insulin pattern is outside the target range and is confirmed based on the glycemic risk, wherein the recommendation is based on the amount the daily insulin pattern is outside the range and at least one of a quantitative assessment of risk and a qualitative assessment of risk. Determining the recommendation is performed when a daily insulin pattern is within the target range and when a glycemic risk fails to meet at least one criteria. The change comprises an increase or a decrease in an amount of at least one of basal insulin, bolus insulin, and total daily insulin. The recommendation is in the form of a report, a command or signal or instructions to an insulin delivery system, or a therapy optimization algorithm.

In an implementation, a method comprises: identifying outlier data in a daily insulin relationship pattern data set; analyzing outlier data in a daily insulin relationship pattern data set; removing the outlier data from the daily insulin relationship pattern data set; and communicating the removal of the outlier data to a diabetes management system.

Implementations may include some or all of the following features. The outlier data comprises inaccurate or misrepresentative data that skews pattern analysis. Analyzing the outlier data comprises analyzing the data set to determine whether at least one of a recommendation and an adjustment can be made. The method further comprises outputting information related to the outlier data removed from the daily insulin relationship pattern data set.

In an implementation, a system comprises: at least one processor; and a non-transitory computer readable medium comprising instructions that, when executed by the at least one processor, cause the system to: identify outlier data in a daily insulin relationship pattern data set; analyze outlier data in a daily insulin relationship pattern data set; remove the outlier data from the daily insulin relationship pattern data set; and communicate the removal of the outlier data to a diabetes management system.

Implementations may include some or all of the following features. The outlier data comprises inaccurate or misrepresentative data that skews pattern analysis. Analyzing the outlier data comprises analyzing the data set to determine whether at least one of a recommendation and an adjustment can be made. The computer readable medium further comprises instructions that, when executed by the at least one processor, cause the system to output information related to the outlier data removed from the daily insulin relationship pattern data set.

In an implementation, a system comprises: an insulin relationship quantifier configured to: identify outlier data in a daily insulin relationship pattern data set; analyze outlier data in a daily insulin relationship pattern data set; and remove the outlier data from the daily insulin relationship pattern data set; and an insulin recommender configured to communicate the removal of the outlier data to a diabetes management system.

Implementations may include some or all of the following features. The outlier data comprises inaccurate or misrepresentative data that skews pattern analysis. Analyzing the outlier data comprises analyzing the data set to determine whether at least one of a recommendation and an adjustment can be made. The insulin recommender is further configured to output information related to the outlier data removed from the daily insulin relationship pattern data set.

In an implementation, a method comprises: identifying a variability in a daily insulin relationship pattern data set; analyzing the variability comprising analyzing data that fall at least one of above, within, or below a target range; and making adjustments to the data set based on the analyzing.

Implementations may include some or all of the following features. Identifying the variability comprises at least one of analyzing individual data points or evaluating a mean or standard deviation against one or more criteria. Identifying the variability comprises evaluating a superset of data to identify one or more subsets of data associated with a different daily insulin pattern. Making adjustments comprises at least one of adjusting definitions of data and dividing data into a plurality of distinct data sets or patterns.

In an implementation, a system comprises: at least one processor; and a non-transitory computer readable medium comprising instructions that, when executed by the at least one processor, cause the system to: identify a variability in a daily insulin relationship pattern data set; analyze the variability comprising analyzing data that fall at least one of above, within, or below a target range; and make adjustments to the data set based on the analyzing.

Implementations may include some or all of the following features. Identifying the variability comprises at least one of analyzing individual data points or evaluating a mean or standard deviation against one or more criteria. Identifying the variability comprises evaluating a superset of data to identify one or more subsets of data associated with a different daily insulin pattern. Making adjustments comprises at least one of adjusting definitions of data and dividing data into a plurality of distinct data sets or patterns.

In an implementation, a system comprises: at least one of an insulin relationship quantifier or an insulin recommender configured to: identify a variability in a daily insulin relationship pattern data set; analyze the variability comprising analyzing data that fall at least one of above, within, or below a target range; and make adjustments to the data set based on the analyzing.

Implementations may include some or all of the following features. Identifying the variability comprises at least one of analyzing individual data points or evaluating a mean or standard deviation against one or more criteria. Identifying the variability comprises evaluating a superset of data to identify one or more subsets of data associated with a different daily insulin pattern. Making adjustments comprises at least one of adjusting definitions of data and dividing data into a plurality of distinct data sets or patterns.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method comprising:
assessing a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk;
quantifying daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period;
determining a recommendation for one or more aspects of daily insulin of a patient based on a target range and the glycemic risk and the daily insulin relationships quantification, wherein the recommendation comprises a change in at least one of basal insulin, bolus insulin, and total daily insulin; and
outputting the recommendation to a diabetes management system.

2. The method of claim 1, further comprising receiving the glucose data and the insulin data for the patient prior to assessing the glycemic risk and quantifying the daily insulin relationships.

3. The method of claim 1, wherein the glycemic risk is a quantification of the risk of current and future hyperglycemia and/or hypoglycemia.

4. The method of claim 1, wherein assessing the glycemic risk uses at least one of prediction and state estimation.

5. The method of claim 1, wherein the glycemic risk is assessed in terms of at least one of sample means, sample variance, time-in-range, episodes of high/low BG (blood glucose), low blood glucose risk, high blood glucose risk, and overall risk.

6. The method of claim 1, further comprising generating a risk profile that illustrates a quantified assessment of glycemic risk.

7. The method of claim 1, wherein the aspects of daily insulin comprise at least one of total daily insulin, total daily basal, or total daily bolus.

8. The method of claim 1, further comprising identifying relationship patterns based on the aspects of daily insulin for each day over a series of days.

9. The method of claim 1, wherein quantifying the daily insulin relationships based on insulin data further comprises calculating at least one of a ratio and a percentage of one aspect of daily insulin relative to another aspect.

10. The method of claim 1, further comprising removing outlier data from at least one of the glucose data and the insulin data prior to at least one of assessing the glycemic risk and quantifying the daily insulin relationships.

11. The method of claim 1, wherein determining the recommendation is performed when a daily insulin pattern is outside the target range, and wherein the recommendation is based on the glycemic risk.

12. The method of claim 1, wherein determining the recommendation is performed when a daily insulin pattern is outside the target range and is confirmed based on the glycemic risk, wherein the recommendation is based on the amount the daily insulin pattern is outside the range and at least one of a quantitative assessment of risk and a qualitative assessment of risk.

13. The method of claim 1, wherein determining the recommendation is performed when a daily insulin pattern is within the target range and when a glycemic risk fails to meet at least one criteria.

14. The method of claim 1, wherein the change comprises an increase or a decrease in an amount of at least one of basal insulin, bolus insulin, and total daily insulin.

15. The method of claim 1, wherein the recommendation is in the form of a report, a command or signal or instructions to an insulin delivery system, or a therapy optimization algorithm.

16. A system comprising:
at least one processor; and
a non-transitory computer readable medium comprising instructions that, when executed by the at least one processor, cause the system to:
assess a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk;
quantify daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period;
determine a recommendation for one or more aspects of daily insulin of a patient based on a target range and the glycemic risk and the daily insulin relationships quantification, wherein the recommendation comprises a change in at least one of basal insulin, bolus insulin, and total daily insulin; and
output the recommendation to a diabetes management system.

17. The system of claim 16, wherein the computer readable medium further comprises instructions that, when executed by the at least one processor, cause the system to receive the glucose data and the insulin data for the patient prior to assessing the glycemic risk and quantifying the daily insulin relationships.

18. The system of claim 16, wherein the glycemic risk is a quantification of the risk of current and future hyperglycemia and/or hypoglycemia.

19. The system of claim 16, wherein assessing the glycemic risk uses at least one of prediction and state estimation.

20. The system of claim 16, wherein the glycemic risk is assessed in terms of at least one of sample means, sample variance, time-in-range, episodes of high/low BG (blood glucose), low blood glucose risk, high blood glucose risk, and overall risk.

21. The system of claim 16, wherein the computer readable medium further comprises instructions that, when executed by the at least one processor, cause the system to generate a risk profile that illustrates a quantified assessment of glycemic risk.

22. The system of claim 16, wherein the aspects of daily insulin comprise at least one of total daily insulin, total daily basal, or total daily bolus.

23. The system of claim 16, wherein the computer readable medium further comprises instructions that, when executed by the at least one processor, cause the system to identify relationship patterns based on the aspects of daily insulin for each day over a series of days.

24. The system of claim 16, wherein quantifying the daily insulin relationships based on insulin data further comprises calculating at least one of a ratio and a percentage of one aspect of daily insulin relative to another aspect.

25. The system of claim 16, wherein the computer readable medium further comprises instructions that, when executed by the at least one processor, cause the system to remove outlier data from at least one of the glucose data and the insulin data prior to at least one of assessing the glycemic risk and quantifying the daily insulin relationships.

26. The system of claim 16, wherein determining the recommendation is performed when a daily insulin pattern is outside the target range, and wherein the recommendation is based on the glycemic risk.

27. The system of claim 16, wherein determining the recommendation is performed when a daily insulin pattern is outside the target range and is confirmed based on the glycemic risk, wherein the recommendation is based on the amount the daily insulin pattern is outside the range and at least one of a quantitative assessment of risk and a qualitative assessment of risk.

28. The system of claim 16, wherein determining the recommendation is performed when a daily insulin pattern is within the target range and when a glycemic risk fails to meet at least one criteria.

29. The system of claim 16, wherein the change comprises an increase or a decrease in an amount of at least one of basal insulin, bolus insulin, and total daily insulin.

30. The system of claim 16, wherein the recommendation is in the form of a report, a command or signal or instructions to an insulin delivery system, or a therapy optimization algorithm.

31. A system comprising:
a glycemic risk assessor configured to assess a glycemic risk based on glucose data of a patient, wherein the glucose data comprises continuous glucose monitoring (CGM) data or flash glucose monitoring (FGM) data received for the patient over a time period, wherein the glycemic risk comprises at least one of a hyperglycemic risk and a hypoglycemic risk;
an insulin relationship quantifier configured to quantify daily insulin relationships based on insulin data, wherein quantifying the daily insulin relationships based on the insulin data comprises calculating a plurality of aspects of daily insulin for each day over the time period and comparing the calculated aspects over the time period, wherein the insulin data comprises basal insulin data and bolus insulin data, wherein the basal insulin data and the bolus insulin data are received for the patient over the time period; and
an insulin recommender configured to determine a recommendation for one or more aspects of daily insulin of a patient based on a target range and the glycemic risk and the daily insulin relationships quantification, wherein the recommendation comprises a change in at least one of basal insulin, bolus insulin, and total daily insulin, and output the recommendation to a diabetes management system.

32. The system of claim 31, wherein the glycemic risk assessor is further configured to receive the glucose data and the insulin data for the patient prior to assessing the glycemic risk and quantifying the daily insulin relationships.

33. The system of claim 31, wherein the glycemic risk is a quantification of the risk of current and future hyperglycemia and/or hypoglycemia.

34. The system of claim 31, wherein assessing the glycemic risk uses at least one of prediction and state estimation.

35. The system of claim 31, wherein the glycemic risk is assessed in terms of at least one of sample means, sample variance, time-in-range, episodes of high/low BG (blood glucose), low blood glucose risk, high blood glucose risk, and overall risk.

36. The system of claim 31, wherein the insulin relationship quantifier or the insulin recommender is further configured to generate a risk profile that illustrates a quantified assessment of glycemic risk.

37. The system of claim 31, wherein the aspects of daily insulin comprise at least one of total daily insulin, total daily basal, or total daily bolus.

38. The system of claim 31, wherein the insulin relationship quantifier is further configured to identify relationship patterns based on the aspects of daily insulin for each day over a series of days.

39. The system of claim 31, wherein quantifying the daily insulin relationships based on insulin data further comprises calculating at least one of a ratio and a percentage of one aspect of daily insulin relative to another aspect.

40. The system of claim 31, wherein the glycemic risk assessor is further configured to remove outlier data from at least one of the glucose data and the insulin data prior to at least one of assessing the glycemic risk and quantifying the daily insulin relationships.

41. The system of claim 31, wherein determining the recommendation is performed when a daily insulin pattern is outside the target range, and wherein the recommendation is based on the glycemic risk.

42. The system of claim 31, wherein determining the recommendation is performed when a daily insulin pattern is outside the target range and is confirmed based on the glycemic risk, wherein the recommendation is based on the amount the daily insulin pattern is outside the range and at least one of a quantitative assessment of risk and a qualitative assessment of risk.

43. The system of claim 31, wherein determining the recommendation is performed when a daily insulin pattern is within the target range and when a glycemic risk fails to meet at least one criteria.

44. The system of claim 31, wherein the change comprises an increase or a decrease in an amount of at least one of basal insulin, bolus insulin, and total daily insulin.

45. The system of claim 31, wherein the recommendation is in the form of a report, a command or signal or instructions to an insulin delivery system, or a therapy optimization algorithm.
